SEQUENCE LISTING (15 sequences)

SEQ ID NO: 1 - DNA, 912 bp, Homo sapiens
  CDS (1)...(912)
atggggaaggaggacactcaagaaactcgcacagagccaaagatgtttgtcttgctctatgttacaagttttgccatttgtgccagtggacaaccccggggtaatcagttgaaaggagagaactactcccccaggtatatctgcagcattcctggcttgcctggacctccagggccccctggagcaaatggttcccctgggccccatggtcgcatcggccttccaggaagagatggtagagacggcaggaaaggagagaaaggtgaaaagggaactgcaggtttgagaggtaagactggaccgctaggtcttgccggtgagaaaggggaccaaggagagactgggaagaaaggacccataggaccagagggagagaaaggagaagtaggtccaattggtcctcctggaccaaagggagacagaggagaacaaggggacccggggctgcctggagtttgcagatgtggaagcatcgtgctcaaatccgccttttctgttggcatcacaaccagctacccagaagaaagactacctattatatttaacaaggtcctcttcaacgagggagagcactacaaccctgccacagggaagttcatctgtgctttcccagggatctattacttttcttatgatatcacattggctaataagcatctggcaatcggactggtacacaatgggcaataccggataaagaccttcgacgccaacacaggaaaccatgatgtggcttcggggtccacagtcatctatctgcagccagaagatgaagtctggctggagattttcttcacagaccagaatggcctcttctcagacccaggttgggcagacagcttattctccgggtttctcttatacgttgacacagattacctagattccatatcagaagatgatgaattgtga

SEQ ID NO: 2 - protein, 303 aa, Homo sapiens
MGKEDTQETRTEPKMFVLLYVTSFAICASGQPRGNQLKGENYSPRYICSIPGLPGPPGPPGANGSPGPHGRIGLPGRDGRDGRKGEKGEKGTAGLRGKTGPLGLAGEKGDQGETGKKGPIGPEGEKGEVGPIGPPGPKGDRGEQGDPGLPGVCRCGSIVLKSAFSVGITTSYPEERLPIIFNKVLFNEGEHYNPATGKFICAFPGIYYFSYDITLANKHLAIGLVHNGQYRIKTFDANTGNHDVASGSTVIYLQPEDEVWLEIFFTDQNGLFSDPGWADSLFSGFLLYVDTDYLDSISEDDEL

SEQ ID NO: 3 - protein, 281 aa, Homo sapiens
MGSRGQGLLLAYCLLLAFASGLVLSRVPHVQGEQQEWEGTEELPSPPDHAERAEEQHEKYRPSQDQGLPASRCLRCCDPGTSMYPATAVPQINITILKGEKGDRGDRGLQGKYGKTGSAGARGHTGPKGQKGSMGAPGERCKSHYAAFSVGRKKPMHSNHYYQTVIFDTEFVNLYDHFNMFTGKFYCYVPGLYFFSLNVHTWNQKETYLHIMKNEEEVVILFAQVGDRSIMQSQSLMLELREQDQVWVRLYKGERENAIFSEELDTYITFSGYLVKHATEP

SEQ ID NO: 4 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 5 - protein, 285 aa, Homo sapiens
MIPWVLLACALPCAADPLLGAFARRDFRKGSPQLVCSLPGPQGPPGPPGAPGPSGMMGRMGFPGKDGQDGHDGDRGDSGEEGPPGRTGNRGKPGPKGKAGAIGRAGPRGPKGVNGTPGKHGTPGKKGPKGKKGEPGLPGPCSCGSGHTKSAFSVAVTKSYPRERLPIKFDKILMNEGGHYNASSGKFVCGVPGIYYFTYDITLANKHLAIGLVHNGQYRIRTFDANTGNHDVASGSTILALKQGDEVWLQIFYSEQNGLFYDPYWTDSLFTGFLIYADQDDPNEV

SEQ ID NO: 6 - protein, 31 aa, Artificial Sequence
  C1q Aromatic Motif
FXXXXXXXXXXXXXXXXXFXXXXXGXYXFXX

SEQ ID NO: 7 - DNA, 18 bp, Artificial Sequence
  Detenerate nucleotide primer
gaysargtntggbtnsar

SEQ ID NO: 8 - DNA, 18 bp, Artificial Sequence
  Degenerate nucleotide primer
cnnggnntntaytaytty

SEQ ID NO: 9 - DNA, 18 bp, Artificial Sequence
  Degenerate nucleotide primer
aaysarsrnrrncaytay

SEQ ID NO: 10 - DNA, 18 bp, Artificial Sequence
  Degenerate nucleotide primer
wsnggnaarttyvhntgy

SEQ ID NO: 11 - DNA, 909 bp, Artificial Sequence
  Degenerate nucleotide encoding the polypeptide of SEQ ID NO2
atgggnaargargayacncargaracnmgnacngarccnaaratgttygtnytnytntaygtnacnwsnttygcnathtgygcnwsnggncarccnmgnggnaaycarytnaarggngaraaytaywsnccnmgntayathtgywsnathccnggnytnccnggnccnccnggnccnccnggngcnaayggnwsnccnggnccncayggnmgnathggnytnccnggnmgngayggnmgngayggnmgnaarggngaraarggngaraarggnacngcnggnytnmgnggnaaracnggnccnytnggnytngcnggngaraarggngaycarggngaracnggnaaraarggnccnathggnccngarggngaraarggngargtnggnccnathggnccnccnggnccnaarggngaymgnggngarcarggngayccnggnytnccnggngtntgymgntgyggnwsnathgtnytnaarwsngcnttywsngtnggnathacnacnwsntayccngargarmgnytnccnathathttyaayaargtnytnttyaaygarggngarcaytayaayccngcnacnggnaarttyathtgygcnttyccnggnathtaytayttywsntaygayathacnytngcnaayaarcayytngcnathggnytngtncayaayggncartaymgnathaaracnttygaygcnaayacnggnaaycaygaygtngcnwsnggnwsnacngtnathtayytncarccngargaygargtntggytngarathttyttyacngaycaraayggnytnttywsngayccnggntgggcngaywsnytnttywsnggnttyytnytntaygtngayacngaytayytngaywsnathwsngargaygaygarytn

SEQ ID NO: 12 - DNA, 18 bp, Artificial Sequence
  Oligonucleotide 23,631
cgagggagagcactacaa

SEQ ID NO: 13 - DNA, 18 bp, Artificial Sequence
  Oligonucleotide 23,632
ttgccagatgcttattag

SEQ ID NO: 14 - DNA, 1282 bp, Mus musculus
  CDS (79)...(945)
ggcacgaggaggaaagatcctgacttttgtacactgggaatcctgcagcaacctaccctcccagaacacgagcccaagatgattgtcctgctctacgtgacgagtcttgccatctgtgcaagtggacaacctcgggccaatcaggctaagggagagagctactctccaaggtacatctgcagcatccctggattacctgggcccccaggtcctcctggagcaaatggctcccctgggccccatggtcgcattggccttcctggaagggatggtagagatggcagaaaaggagagaagggggaaaagggcactgcaggtctaaaaggtaaaactggacccctgggccttgctggtgagaaaggagaccaaggagaaactgggaagaaaggacccataggaccagagggtgagaaaggagaagtcggtccagctgggcctcctgggccaaagggagacagaggagatcaaggggacccagggctgcctggagtgtgcaggtgtggaagcattgtgctcaaatctgccttttcagttggcatcacaaccagctacccagaagaaagactacccatcatatttaacaaagtcctcttcaatgagggggagcattacaaccctgcaacggggaagttcatttgcgctttcccagggatctattacttttcttatgacatcacgttggccaataagcacctagcaatcgggctggtgcacaatgggcagtaccggataaggacctttgatgccaacacagggaaccatgatgtggcatcggggtccacagtcatctacctgcagccagaagatgaggtctggctggagatcttcttcaatgaccagaacggcctcttctcggatccaggctgggcagacagcttgttctctgggtttctcctctatgtcgatacagattacctggattctatatcagaggatgatgagctgtgatccagaccactacaggcctgaatgttgcaaacatgagtaccacagtggctgacactctaatctggagtgctggaaggtggagcaagtgatacggggattcagaaaacgttttttacagacgactcaggctgagttatcaaaataagacaaaccaccaactagctgaaatcacaacaaaacgaatggcatacaataacctcagacatggaccccctaaagtaatgatcctaaatattgaagcaaattaaagcaaatgatgttaacaaatttgaatgcccttggcaatacaaccagctggaaatgacactgcctcattaaatattcataaaacccc

SEQ ID NO: 15 - protein, 289 aa, Mus musculus
MIVLLYVTSLAICASGQPRANQAKGESYSPRYICSIPGLPGPPGPPGANGSPGPHGRIGLPGRDGRDGRKGEKGEKGTAGLKGKTGPLGLAGEKGDQGETGKKGPIGPEGEKGEVGPAGPPGPKGDRGDQGDPGLPGVCRCGSIVLKSAFSVGITTSYPEERLPIIFNKVLFNEGEHYNPATGKFICAFPGIYYFSYDITLANKHLAIGLVHNGQYRIRTFDANTGNHDVASGSTVIYLQPEDEVWLEIFFNDQNGLFSDPGWADSLFSGFLLYVDTDYLDSISEDDEL

(19) United States
(12) Patent Application Publication
(10) Pub. No.: US 2003/0129698 A1
(43) Pub. Date: Jul. 10, 2003

(54) Adipocyte complement related protein homolog zacrp7

(75) Inventors: Christopher S. Piddington, Thousand Oaks, CA (US); Paul O. Sheppard, Granite Falls, WA (US)

Correspondence Address: Brian J. Walsh, Patent Department, ZymoGenetics, Inc., 1201 Eastlake Avenue East, Seattle, WA, 98102, US

(73) Assignee: ZymoGenetics, Inc.

(21) Appl. No.: 10/234,000

(22) Filed: Aug. 30, 2002

Related U.S. Application Data
(63) Continuation of application No. 09/577,298, filed on May 23, 2000, now abandoned.
(60) Provisional application No. 60/158,448, filed on Oct. 7, 1999.
(60) Provisional application No. 60/145,589, filed on Jul. 26, 1999.
(60) Provisional application No. 60/136,289, filed on May 27, 1999.

(51) Int. Cl. C07K 14/78; C12P 21/02; C12N 5/06; C07H 21/04

(52) U.S. Cl. 435/69.1; 435/320.1; 435/325; 536/23.2; 530/356

(57) ABSTRACT

The present invention relates to polynucleotide and polypeptide molecules for zacrp7, a novel member of the family of proteins bearing a collagen-like domain and a C1q domain. The polypeptides and polynucleotides encoding them, are involved in homo and heterotrimerization or oligomerization and may be used in the study thereof. The present invention also includes antibodies to the zacrp7 polypeptides.

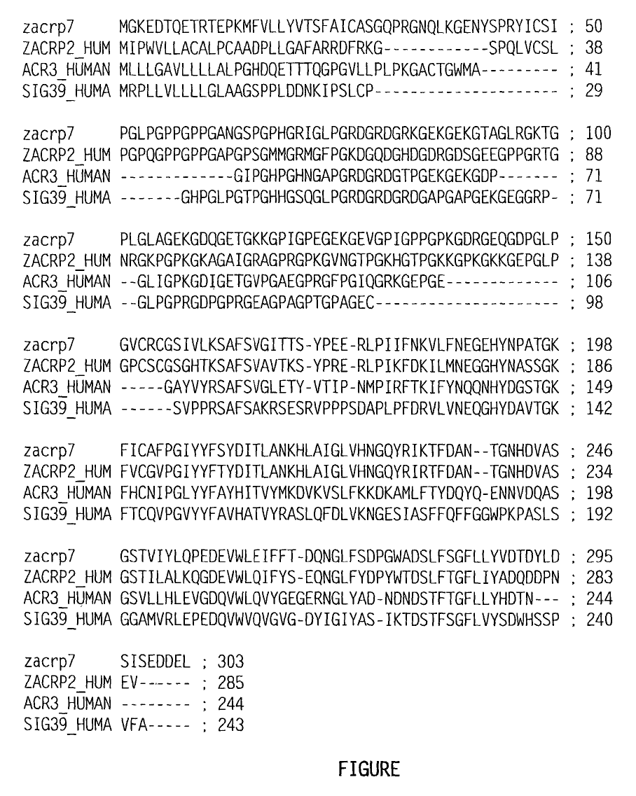

### REFERENCE TO RELATED APPLICATIONS

This application is related to Provisional Application No. 60/136,289, filed on May 27, 1999; No. 60/145,589, filed on Jul. 26, 1999; and No. 60/158,448, filed on Oct. 7, 1999. Under 35 U.S.C. §119(e)(1), this application claims benefit of said Provisional Applications.

### BACKGROUND OF THE INVENTION

Cell-cell and cell-extracellular matrix interactions allow for exchange of information between, and coordination among, various cells of a multi-cellular organism and are fundamental for most biological processes. These interactions play a role in everything from fertilization to death. Such interactions are essential during development and differentiation and are critical for the function and protection of the organism. For example, interaction between the cell and its environment is necessary to initiate and mediate tissue remodeling. Tissue remodeling may be initiated, for example, in response to many factors including physical injury, cytotoxic injury, metabolic stress or developmental stimuli. Modulation between pathology and healing (or metabolic optimization) may be done, in part, by the interaction of stimulated cells with the extracellular matrix as well as the local solvent.

A family of proteins that plays a role in the interaction of cells with their environment, and appear to act at the interface of the extracellular matrix and the cell, are the adipocyte complement related proteins. These proteins include, Acrp30, a 247 amino acid polypeptide that is expressed exclusively by adipocytes. The Acrp30 polypeptide is composed of a amino-terminal signal sequence, a 27 amino acid stretch of no known homology, 22 perfect Gly-Xaa-Pro or imperfect Gly-Xaa-Xaa collagen repeats and a carboxy terminal globular domain. See, Scherer et al., J. Biol. Chem. 270(45): 26746-9, 1995 and International Patent Application No. WO 96/39429. Acrp30, an abundant human serum protein regulated by insulin, shares structural similarity, particularly in the carboxy-terminal globular domain, to complement factor Clq and to a summer serum protein of hibernating Siberian chipmunks (Hib27). Expression of Acrp30 is induced over 100-fold during adipocyte differentiation. Acrp30 is suggested for use in modulating energy balance and in identifying adipocytes in test samples.

Additional members include zsig37 (WO 99/04000), a 281 amino acid residue protein expressed predominantly in heart, aorta and placenta, having 14 collagen repeats and a C1q globular domain similar to ACRP30. Zsig37 has been shown to inhibit complement activity, binds to SK5 fibroblasts and stimulates proliferation at concentrations known to initiate Clq-cell responses. Zsig37 also specifically inhibits collagen activation of platelets in human whole blood and platelet rich plasma in a dose dependent manner (copending U.S. patent application Ser. No. 09/253,604). Also included is zsig39 (WO 99/10492), a 243 amino acid residue protein expressed predominantly in heart and small intestine, having 22 or 23 collagen repeats and a C1q domain similar to ACRP30 and zsig37.

These proteins all share a C1q domain. Complement factor Clq consists of six copies of three related polypeptides (A, B and C chains), with each polypeptide being about 225 amino acids long with a near amino-terminal collagen domain and a carboxy-terminal globular region. Six triple helical regions are formed by the collagen domains of the six A, six B and six C chains, forming a central region and six stalks. A globular head portion is formed by association of the globular carboxy terminal domain of an A, a B and a C chain. Clq is therefore composed of six globular heads linked via six collagen-like stalks to a central fibril region. Sellar et al., Biochem. J. 274: 481-90, 1991. This configuration is often referred to as a bouquet of flowers. Acrp30 has a similar bouquet structure formed from a single type of polypeptide chain. The Clq globular domain of ACRP30 has been determined to have a 10 beta strand “jelly roll” topology (Shapiro and Scherer, Curr. Biol. 8:335-8, 1998). The structural elements such as folding topologies, conserved residues and similar trimer interfaces and intron positions are homologous to the tumor necrosis factor family suggesting a link between the TNF and C1q families. Zsig39 and zsig37 share this structure and homology as well.

Proteins that play a role in cellular interaction, such as transcription factors and hormones are useful diagnostic and therapeutic agents. Proteins that mediate specific interactions, such a remodeling, would be particularly useful. The present invention provides such polypeptides for these and other uses that should be apparent to those skilled in the art from the teachings herein.

### SUMMARY OF THE INVENTION

Within one aspect, the invention provides an isolated polypeptide comprising a sequence of amino acid residues that is at least 80% identical in amino acid sequence to residues 52-303 of SEQ ID NO:2, wherein said sequence comprises: Gly-Xaa-Xaa and Gly-Xaa-Pro repeats forming a collagen-like domain, wherein Xaa is any amino acid residue; and a carboxyl-terminal C1q domain. Within one embodiment the polypeptide is at least 90% identical in amino acid sequence to residues 31-303 of SEQ ID NO:2. Within a related embodiment any differences between said polypeptide and SEQ ID NO:2 are due to conservative amino acid substitutions. Within another embodiment the collagen-like domain consists of 26 Gly-Xaa-Xaa repeats and 8 Gly-Xaa-Pro repeat. Within yet another embodiment the polypeptide comprises: an amino terminal region; 26 Gly-Xaa-Xaa repeats and 8 Gly-Xaa-Pro repeat forming a collagen-like domain, wherein Xaa is any amino acid residue; and a carboxyl-terminal Clq domain comprising 10 beta strands corresponding to amino acid residues 164-168, 184-186, 192-195, 199-201, 205-216, 220-226, 231-238, 241-253, 258-263 and 281-285 of SEQ ID NO:2. Within a further embodiment the polypeptide specifically binds with an antibody that specifically binds with a polypeptide of SEQ ID NO:2. Within another embodiment the collagen-like domain comprises amino acid residues 52-153 of SEQ ID NO:2. Within another embodiment the Clq domain comprises amino acid residues 154-303 of SEQ ID NO:2. Within other embodiments the polypeptide comprises residues 52-303 of SEQ ID NO:2, residues 31-303 of SEQ ID NO:2 or 1-303 of SEQ ID NO:2. Within another embodiment the polypeptide is complexed by intermolecular disulfide bonds to form a homotrimer. Within yet another embodiment the polypeptide is complexed by intermolecular disulfide bonds, to one or more polypeptides having a collagen-like domain, to form a heterotrimer. Within a further embodiment the polypeptide is covalently linked at the amino or carboxyl terminus to a moiety selected from the group consisting of affinity tags, toxins, radionucleotides, enzymes and fluorophores.

The invention also provided an isolated polypeptide selected from the group consisting of: a) a polypeptide consisting of a sequence of amino acid residues from residue 52 to residue 153 of SEQ ID NO:2; and b) a polypeptide consisting of a sequence of amino acid residues from residue 154 to residue 303 of SEQ ID NO:2.

Within another aspect the invention provides a fusion protein consisting essentially of a first portion and a second portion joined by a peptide bond, said first portion consisting of a polypeptide selected from the group consisting of: a) polypeptide comprising a sequence of amino acid residues that is at least 80% identical in amino acid sequence to residues 52-303 of SEQ ID NO:2, wherein said sequence comprises: Gly-Xaa-Xaa and Gly-Xaa-Pro repeats forming a collagen-like domain, wherein Xaa is any amino acid residue; and a carboxyl-terminal C1q domain; b) polypeptide comprising: an amino terminal region; 26 Gly-Xaa-Xaa repeats and 8 Gly-Xaa-Pro repeat forming a collagen-like domain, wherein Xaa is any amino acid residue; and a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2; c) a portion of the zacrp7 polypeptide as shown in SEQ ID NO:2, comprising the collagen-like domain or a portion of the collagen-like domain capable of trimerization or oligomerization; d) a portion of the zacrp7 polypeptide as shown in SEQ ID NO:2, comprising the C1q domain or an active portion of the C1q domain; or e) a portion of the zacrp7 polypeptide as shown in SEQ ID NO:2 comprising of the collagen-like domain and the C1q domain; and said second portion comprising another polypeptide. Within a related embodiment the first portion is selected from the group consisting of: a) a polypeptide consisting of the sequence of amino acid residue 52 to amino acid residue 153 of SEQ ID NO:2; b) a polypeptide consisting of the sequence of amino acid residue 154 to amino acid residue 303 of SEQ ID NO:2; c) a polypeptide consisting of the sequence of amino acid residue 52 to 303 of SEQ ID NO:2; d) a polypeptide consisting of the sequence of amino acid residue 31 to 303 of SEQ ID NO:2; and e) a polypeptide consisting of the sequence of amino acid residue 1 to 303 of SEQ ID NO:2.

The invention also provides a polypeptide as described above; in combination with a pharmaceutically acceptable vehicle.

Within another aspect the invention provides a method of producing an antibody to a polypeptide comprising: inoculating an animal with a polypeptide selected from the group consisting of: a) polypeptide comprising a sequence of amino acid residues that is at least 80% identical in amino acid sequence to residues 52-303 of SEQ ID NO:2, wherein said sequence comprises: Gly-Xaa-Xaa and Gly-Xaa-Pro repeats forming a collagen-like domain, wherein Xaa is any amino acid residue; and a carboxyl-terminal C1q domain; b) polypeptide comprising: an amino terminal region; 26 Gly-Xaa-Xaa repeats and 8 Gly-Xaa-Pro repeat forming a collagen-like domain, wherein Xaa is any amino acid residue; and a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2; c) a portion of the zacrp7 polypeptide as shown in SEQ ID NO:2, comprising the collagen-like domain or a portion of the collagen-like domain capable of trimerization or oligomerization; d) a portion of the zacrp7 polypeptide as shown in SEQ ID NO:2, comprising the Clq domain or an active portion of the C1q domain; or e) a portion of the zacrp7 polypeptide as shown in SEQ ID NO:2 comprising of the collagen-like domain and the Clq domain; and wherein said polypeptide elicits an immune response in the animal to produce the antibody; and isolating the antibody from the animal.

Also provides are antibodies or antibody fragments that specifically binds to a polypeptide as described above. Within one embodiment the antibody is selected from the group consisting of: a) polyclonal antibody; b) murine monoclonal antibody; c) humanized antibody derived from b); and d) human monoclonal antibody. Within another embodiment the antibody fragment is selected from the group consisting of F(ab′), F(ab), Fab′, Fab, Fv, scFv, and minimal recognition unit. Within another embodiment is provided an anti-idiotype antibody that specifically binds to the antibody described above. Also provided by the invention is a binding protein that specifically binds to an epitope of a polypeptide as described above.

Within another aspect the invention provides an isolated polynucleotide encoding a polypeptide as described above. Also provided herein is an isolated polynucleotide selected from the-group consisting of: a) a sequence of nucleotides from nucleotide 1 to nucleotide 909 of SEQ ID NO:1; b) a sequence of nucleotides from nucleotide 91 to nucleotide 909 of SEQ ID NO:1; c) a sequence of nucleotides from nucleotide 91 to nucleotide 459 of SEQ ID NO:1; d) a sequence of nucleotides from nucleotide 154 to nucleotide 909 of SEQ ID NO:1; e) a sequence of nucleotides from nucleotide 154 to nucleotide 459 of SEQ ID NO:1; f) a sequence of nucleotides from nucleotide 460 to nucleotide 909 of SEQ ID NO:1; g) a polynucleotide encoding a polypeptide consisting of the amino acid sequence of residues 52 to 153 of SEQ ID NO:2; h) a polynucleotide encoding a polypeptide consisting of the amino acid sequence of residues 154 to 303 of SEQ ID NO:2; i) a polynucleotide that remains hybridized, following stringent wash conditions, to a polynucleotide consisting of the nucleotide sequence of SEQ ID NO:1, or the complement of SEQ ID NO:1; j) nucleotide sequences complementary to a), b), c), d), e), f), g), h) or i) and k) degenerate nucleotide sequences of g) or h).

Also provided is an isolated polynucleotide encoding a fusion protein as described above.

The invention also provided an isolated polynucleotide consisting of the sequence of nucleotide 1 to nucleotide 909 of SEQ ID NO:11.

Within another aspect the invention provides an expression vector comprising the following operably linked elements: a transcription promoter; a DNA segment encoding a polypeptide as described above; and a transcription terminator. Within one embodiment the DNA segment further encodes a secretory signal sequence operably linked to said polypeptide. Within a related embodiment the secretory signal sequence comprises residues 1-30 of SEQ ID NO:2.

The invention also provides a cultured cell into which has been introduced an expression vector as described above, wherein said cell expresses said polypeptide encoded by said DNA segment. Within one embodiment the cultured cell further includes one or more expression vectors comprising DNA segments encoding polypeptides having collagen-like domains.

Within another aspect the invention provides a method of producing a protein comprising: culturing a cell into which has been introduced an expression vector as described above; whereby said cell expresses said protein encoded by said DNA segment; and recovering said expressed protein. Within one embodiment the expressed protein is a homotrimer. Within another embodiment the expressed protein is a heterotrimer.

Within another aspect the invention provides a method of detecting the presence of zacrp7 gene expression in a biological sample, comprising:(a) contacting a zacrp7 nucleic acid probe under hybridizing conditions with either (i) test RNA molecules isolated from the biological sample, or (ii) nucleic acid molecules synthesized from the isolated RNA molecules, wherein the probe consists of a nucleotide sequence comprising a portion of the nucleotide sequence of the nucleic acid molecule as described above, or complements thereof, and (b) detecting the formation of hybrids of the nucleic acid probe and either the test RNA molecules or the synthesized nucleic acid molecules, wherein the presence of the hybrids indicates the presence of zacrp7 RNA in the biological sample.

Within another aspect is provided a method of detecting the presence of zacrp7 in a biological sample, comprising: (a) contacting the biological sample with an antibody, or an antibody fragment, as described above, wherein the contacting is performed under conditions that allow the binding of the antibody or antibody fragment to the biological sample, and (b) detecting any of the bound antibody or bound antibody fragment.

### BRIEF DESCRIPTION OF THE DRAWING

The FIGURE illustrates a multiple alignment of and zacrp7 polypeptide of the present invention and human ACRP30 (ACR3_HUMAN) (SEQ ID NO:4, Maeda et al., Biochem. Biophys. Res. Commun. 221:286-9, 1996), adipocyte complement related protein homolog zsig39 (SEQ ID NO:3, WO 99/10492) and human adipocyte complement related protein homolog zacrp2 (SEQ ID NO:5, co-pending US Provisional Patent Application No:60/130,207). The multiple alignment performed using a Clustalx multiple alignment tool with the default settings: Blosum Series Weight Matricies, Gap Opening penalty:10.0, Gap Extension penalty:0.05. Multiple alignments were further hand tuned before computing percent identity.

### DETAILED DESCRIPTION OF THE INVENTION

Prior to setting forth the invention in detail, it may be helpful to the understanding thereof to define the following terms.

The term “affinity tag” is used herein to denote a peptide segment that can be attached to a polypeptide to provide for purification or detection of the polypeptide or provide sites for attachment of the polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), substance P, Flag™ peptide (Hopp et al., Biotechnoloqy 6:1204-10, 1988; available from Eastman Kodak Co., New Haven, Conn.), streptavidin binding peptide, or other antigenic epitope or binding domain. See, in general Ford et al., Protein Expression and Purification 2: 95-107, 1991. DNAs encoding affinity tags are available from commercial suppliers (e.g., Pharmacia Biotech, Piscataway, N.J.).

The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal [symbol] and [symbol] carboxyl-terminal” are used herein to denote positions within polypeptides and proteins. Where the context allows, these terms are used with reference to a particular sequence or portion of a polypeptide or protein to denote proximity or relative position. For example, a certain sequence positioned carboxyl-terminal to a reference sequence within a protein is located proximal to the carboxyl terminus of the reference sequence, but is not necessarily at the carboxyl terminus of the complete protein.

The term “biological sample” denotes a sample that is derived from or contains cells, cell components or cell products, including, but not limited to, cell culture supernatants, cell lysates, cleared cell lysates, cell extracts, tissue extracts, blood plasma, serum, and fractions thereof, from a patient.

The term “complement/anti-complement pair [symbol] denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGCTTAGCTT-3′ are 5′-TAGCTTgagtct-3′ and 3′-gtcgacTACCGA-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” denotes a DNA molecule, linear or circular, that comprises a segment encoding a polypeptide of interest operably linked to additional segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985). Another example of an isolated nucleic acid molecule is a chemically-synthesized nucleic acid molecule that is not integrated in the genome of an organism. An isolated nucleic acid molecule that has been isolated from a chromosome of a particular species is smaller than the complete DNA molecule of that chromosome.

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, denotes that the segments are arranged so that they function in concert for their intended purposes, e.g. transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” denotes a portion of a gene containing DNA sequences that provide for the binding of RNA polymerase and initiation of transcription. Promoter sequences are commonly, but not always, found in the 5′ non-coding regions of genes.

The term “receptor” denotes a cell-associated protein that binds to a bioactive molecule (i.e., a ligand) and mediates the effect of the ligand on the cell. Membrane-bound receptors are characterized by a multi-domain structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. Most nuclear receptors also exhibit a multi-domain structure, including an amino-terminal, transactivating domain, a DNA binding domain and a ligand binding domain. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

A “soluble receptor” is a receptor polypeptide that is not bound to a cell membrane. Soluble receptors are most commonly ligand-binding receptor polypeptides that lack transmembrane and cytoplasmic domains. Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Receptor polypeptides are said to be substantially free of transmembrane and intracellular polypeptide segments when they lack sufficient portions of these segments to provide membrane anchoring or signal transduction, respectively.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that has homology with adipocyte complement related protein homolog, zacrp2 (SEQ ID NO:5) (co-pending, co-owned U.S. patent application Ser. No. 09/552,204). The DNA sequence encodes a polypeptide having an amino-terminal signal sequence, an adjacent N-terminal region of non-homology, a collagen domain composed of 34 collagen repeats and a carboxy-terminal globular-like C1q domain. The general polypeptide structure set forth above is shared by zsig39 and Acrp3O (see FIGURE). Other regions of homology, found in the carboxy-terminal globular C1q domain in the aligned proteins, are identified herein as useful primers for searching for other family members. Intra-chain disulfide bonding may involve the cysteines at residues 48, 153, 155 and 201 of SEQ ID NO:2.

The novel zacrp7 polynucleotide of the present invention was initially identified by querying an EST database proteins characterized by a signal sequence, a collagen-like domain and a C1q domain. Polypeptides corresponding to ESTs meeting those search criteria were compared to known sequences to identify proteins having homology to zsig39. An assembled EST cluster was discovered and predicted to be a secreted protein. To identify the corresponding cDNA in various tissues, probes and/or primers are provided herein and can be designed from sequences disclosed, such as SEQ ID NO:1. Tissues expressing zacrp6 could be identified either through hybridization (Northern Blots) or by reverse transcriptase (RT) PCR. Libraries are then generated from tissues which appear to show expression of zacrp7. Single clones from such libraries are then identified through hybridization with the probes and/or by PCR with the primers as described herein. Conformation of the zacrp7 cDNA sequence can be verified using the sequences provided herein. The 912 bp nucleotide sequence is disclosed in SEQ ID NO:1.

Percent identity at the amino acid level over the whole molecule between zacrp7 and other family members is shown in Table 1A. The percent identity over the Clq domain only is shown in Table 1B. The alignments were performed using a Clustalx multiple alignment tool with the default settings: Blosum Series Weight Matricies, Gap Opening penalty:10.0, Gap Extension penalty:0.05. Multiple alignments were further hand tuned before computing percent identity. Percent identity is the total number of identical residues over the length of the overlap.

TABLE 1A

| | zacrp7 | zacrp2 | ACRP30 | zsig39 |
|---|---|---|---|---|
| zacrp7 | 100.0 | 57.2 | 41.1 | 37.4 |
| zacrp2 | 57.2 | 100.0 | 36.9 | 38.3 |
| ACRP30 | 41.4 | 36.9 | 100.0 | 37.0 |
| zsig39 | 37.4 | 38.3 | 37.0 | 100.0 |

TABLE 1B

| | zacrp7 | zacrp2 | ACRP30 | zsig39 |
|---|---|---|---|---|
| zacrp7 | 100.0 | 70.1 | 42.2 | 35.2 |
| zacrp2 | 70.1 | 100.0 | 43.0 | 35.4 |
| ACRP30 | 42.2 | 43.0 | 100.0 | 39.3 |
| zsig39 | 35.2 | 35.4 | 39.3 | 100.0 |

The nucleotide sequence of zacrp7 is described in SEQ ID NO:1, and its deduced amino acid sequence is described in SEQ ID NO:2. As described generally above, the zacrp7 polypeptide includes a signal sequence, ranging from amino acid 1 (Met) to amino acid residue 30 (Gly) of SEQ ID NO:2, nucleotides 1-30 of SEQ ID NO:1. The mature polypeptide therefore ranges from amino acid 31 (Gln) to amino acid 303 (Leu) of SEQ ID NO:2, nucleotides 91 to 909 of SEQ ID NO:1. Within the mature polypeptide, an N-terminal region of no known homology is found, ranging between amino acid residue 31 (Gln) and 50 (Pro) of SEQ ID NO:2, nucleotides 91-153 of SEQ ID NO:1. In addition, a collagen-like domain is found between amino acid 51 (Gly) and 153 (Cys) of SEQ ID NO:2, nucleotides 154 to 459 of SEQ ID NO:1. In the collagen-like domain, 8 perfect Gly-Xaa-Pro and 26 imperfect Gly-Xaa-Xaa repeats are observed. Acrp30 contains 22 perfect or imperfect repeats, zsig39 has 22 or 23 repeats and zacrp2 has 34. Proline residues found in this domain at amino acid residue 54, 57, 66, 75, 135, 147 and 150 of SEQ ID NO:2 may be hydroxylated. The zacrp7 polypeptide also includes a carboxy-terminal C1q domain, ranging from about amino acid 154 (Arg) to 303 (Leu) of SEQ ID NO:2, nucleotides 460 to 909 of SEQ ID NO:1. There is a fair amount of conserved structure within the C1q domain to enable proper folding. An imperfect C1q aromatic motif (F-X(5)-[ND]-X(4)-[FYWL]-X(6)-F-X(5)-G-X-Y-X-F-X-[FY] (SEQ ID NO:6) is found between residues 181 (Phe) and 211 (Tyr) of SEQ ID NO:2. X represents any amino acid residue and the number in parentheses ( ) indicates the amino acid number of residues. The amino acid residues contained within the square parentheses [ ] restrict the choice of amino acid residues at that particular position. Zacrp7 polypeptide, human zsig39, human zacrp2 and Acrp30 appear to be homologous within the collagen domain and in the C1q domain, but not in the N-terminal portion of the mature polypeptide (see FIGURE).

Another aspect of the present invention includes zacrp7 polypeptide fragments. Preferred fragments include those containing the collagen-like domain of zacrp7 polypeptides, ranging from amino acid 1 (Met), 31 (Gln) or 51 (Gly) to amino acid 153 (Cys) of SEQ ID NO:2, a portion of the zacrp7 polypeptide containing the collagen-like domain or a portion of the collagen-like domain capable of dimerization or oligomerization. As used herein the term “collagen” or “collagen-like domain [symbol] refers to a series of repeating triplet amino acid sequences, “repeats” or “collagen repeats” represented by the motifs Gly-Xaa-Pro or Gly-Xaa-Xaa, where Xaa is any amino acid reside. Such domains may contain as many as 34 collagen repeats or more. Moreover, such fragments or proteins containing such collagen-like domains may form heteromeric constructs, usually trimers. Structural analysis and homology to other collagen-like domain containing proteins indicates that zacrp7 polypeptides, fragments or fusions comprising the collagen-like domain can complex with other collagen domain containing polypeptides to form homotrimers and heterotrimers.

These collagen-like domain containing fragments are particularly useful in the study of collagen trimerization or oligomerization or in formation of fusion proteins as described more fully below. Polynucleotides encoding such fragments are also encompassed by the present invention, including the group consisting of (a) polynucleotide molecule comprising a sequence of nucleotides as shown in SEQ ID NO:1 from nucleotide 1, 91 or 154 to nucleotide 459; (b) polynucleotide molecules that encode a zacrp7 polypeptide fragment that is at least 80% identical to the amino acid sequence of SEQ ID NO:2 from amino acid residue 51 (Gly) to amino acid residue 153 (Cys); (c) molecules complementary to (a) or (b); and (d) degenerate nucleotide sequences encoding a zacrp7 polypeptide collagen-like domain fragment.

Other collagen-like domain containing polypeptides include members of the adipocyte complement related protein family, such as zsig37, zsig39 and ACRP30, for example. The trimeric proteins of the present invention are formed by intermolecular disulfide bonds formed between conserved cysteine residues within the polypeptides. The present invention therefore provides zacrp6 polypeptides complexed by intermolecular disulfide bonds to form homotrimers. The invention further provides zacrp6 polypeptides complexed by intermolecular disulfide bonds to other polypeptides having a collagen-like domain, to form heterotrimers.

Other preferred fragments include the globular Clq domain of zacrp7 polypeptides, ranging from amino acid 154 (Arg) to 303 (Leu) of SEQ ID NO:2, nucleotides 460-909 of SEQ ID NO:1, a portion of the zacrp7 polypeptide containing the C1q domain or an active portion of the C1q domain. Other Clq domain containing proteins include zsig37 (WO 99/04000), zsig39 (WO 99/10492), Clq A, B and C (Sellar et al., ibid., Reid, ibid., and Reid et al., Biochem. J. 203: 559-69, 1982), chipmunk hibernation-associated plasma proteins HP-20, HP-25 and HP-27 (Takamatsu et al., Mol. Cell. Biol. 13: 1516-21, 1993 and Kondo & Kondo, J. Biol. Chem. 267: 473-8, 1992), human precerebellin (Urade et al., Proc. Natl. Acad. Sci. USA 88:1069-73, 1991), human endothelial cell multimerin (Hayward et al., J. Biol. Chem. 270:18246-51, 1995) and vertebrate collagens type VIII and X (Muragaki et al., Eur. J. Biochem. 197:615-22, 1991).

The globular C1q domain of ACRP30 has been determined to have a 10 beta strand “jelly roll” topology (Shapiro and Scherer, Curr. Biol. 8:335-8, 1998) that shows significant structural homology to the TNF family and the zacrp7 sequence as represented by SEQ ID NO:2 contains all 10 beta-strands of this structure (amino acid residues 164-168, 184-186, 192-195, 199-201, 205-216, 220-226, 231-238, 241-253, 258-263 and 281-285 of SEQ ID NO:2). These strands have been designated “A”, “A′”, “B”, “B′”, “C”, “D”, “E”, “F”, “G” and “H” respectively.

Zacrp7 has two receptor binding loops, at amino acid residues 168-194 and 225-238. Amino acid residues 205 (Gly), 207 (Tyr), 253 (Leu) and 283 (Gly) appear to be conserved across the superfamily including CD40, TNFα, TNFβ, ACRP30 and zacrp7.

These fragments are particularly useful in the study or modulation of cellular and extracellular matrix interactions. Anti-microbial activity may also be present in such fragments. The homology to TNF proteins suggests such fragments would be useful in obesity-related insulin resistance, immune regulation, inflammatory response, apoptosis and osteoclast maturation. Polynucleotides encoding such fragments are also encompassed by the present invention, including the group consisting of (a) polynucleotide molecules comprising a sequence of nucleotides as shown in SEQ ID NO:1 from nucleotide 460 to nucleotide 909; (b) polynucleotide molecules that encode a zacrp7 polypeptide fragment that is at least 80% identical to the amino acid sequence of SEQ ID NO:2 from amino acid residue 154 (Arg) to amino acid residue 303 (Leu); (c) molecules complementary to (a) or (b); and (d) degenerate nucleotide sequences encoding a zacrp7 polypeptide C1q domain fragment.

Other zacrp7 polypeptide fragments of the present invention include both the collagen-like domain and the C1q domain ranging from amino acid residue 51 (Gly) to 303 (Leu) of SEQ ID NO:2. Polynucleotides encoding such fragments are also encompassed by the present invention, including the group consisting of (a) polynucleotide molecules comprising a sequence of nucleotides as shown in SEQ ID NO:1 from nucleotide 154 to nucleotide 909; (b) polynucleotide molecules that encode a zacrp7 polypeptide fragment that is at least 80% identical to the amino acid sequence of SEQ ID NO:2 from amino acid residue 51 (Gly) to amino acid residue 303 (Leu); (c) molecules complementary to (a) or (b); and (d) degenerate nucleotide sequences encoding a zacrp7 polypeptide collagen-like domain-C1q domain fragment.

The highly conserved amino acids, particularly those in the carboxy-terminal C1q domain of the zacrp7 polypeptide, can be used as a tool to identify new family members. For instance, reverse transcription-polymerase chain reaction (RT-PCR) can be used to amplify sequences encoding the conserved motifs from RNA obtained from a variety of tissue sources. In particular, highly degenerate primers and their complements designed from conserved sequences are useful for this purpose. In particular, the following primers are useful for this purpose:

Degenerate primer sequence encoding amino
acid residues
257-262 of SEQ ID NO:2

| | |
|---|---|
| GAY SAR GTN TGG BTN SAR | (SEQ ID NO:7) |

Degenerate primer sequence encoding amino
acid residues
204-209 of SEQ ID NO:2

| | |
|---|---|
| CNN GON NTN TAY TAY TTY | (SEQ ID NO:8) |

Degenerate primer sequence encoding amino
acid residues
187-192 of SEQ ID NO:2

| | |
|---|---|
| AAY SAR SRN RRN CAY TAY | (SEQ ID NO:9) |

Degenerate primer sequence encoding amino
acid residues
196-201 of SEQ ID NO:2

| | |
|---|---|
| WSN GGN AAR TTY VHN TGY | (SEQ ID NO:10) |

Probes corresponding to complements of the polynucleotides set forth above are also encompassed.

The present invention also provides a zacrp7 murine ortholog (SEQ ID NO:15) and the polynucleotide encoding it (SEQ ID NO:14). The murine homolog shares 96.5% identity at the amino acid level.

The present invention also provides polynucleotide molecules, including DNA and RNA molecules, that encode the zacrp7 polypeptides disclosed herein. Those skilled in the art will readily recognize that, in view of the degeneracy of the genetic code, considerable sequence variation is possible among these polynucleotide molecules. SEQ ID NO:11 is a degenerate DNA sequence that encompasses all DNAs that encode the zacrp7 polypeptide of SEQ ID NO:2. Those skilled in the art will recognize that the degenerate sequence of SEQ ID NO:11 also provides all RNA sequences encoding SEQ ID NO:2 by substituting U for T. Thus, zacrp7 polypeptide-encoding polynucleotides comprising nucleotide 1 to nucleotide 909 of SEQ ID NO:11 and their RNA equivalents are contemplated by the present invention. Table 2 sets forth the one-letter codes used within SEQ ID NO:11 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 2

| Nucleotide | Resolution | Complement | Resolution |
|---|---|---|---|
| A | A | T | I |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:11, encompassing all possible codons for a given amino acid, are set forth in Table 3.

TABLE 3

| | One | | |
|---|---|---|---|
| Amino | Letter | | Degenerate |
| Acid | Code | Codons | Codon |
| Cys | C | IGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 3). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:11 serves as a template for optimizing expression of polynucleotides in various cell types and species commonly used in the art and disclosed herein. Sequences containing preferential codons can be tested and optimized for expression in various species, and tested for functionality as disclosed herein.

The present invention further provides variant polypeptides and nucleic acid molecules that represent counterparts from other species (orthologs). These species include, but are not limited to mammalian, avian, amphibian, reptile, fish, insect and other vertebrate and invertebrate species. Of particular interest are zacrp7 polypeptides from other mammalian species, including murine, porcine, ovine, bovine, canine, feline, equine, and other primate polypeptides. The invention provides a murine ortholog (SEQ ID NO:15) to human zacrp7 (SEQ ID NO:2). Orthologs of human zacrp7 can be cloned using information and compositions provided by the present invention in combination with conventional cloning techniques. For example, a cDNA can be cloned using mRNA obtained from a tissue or cell type that expresses zacrp7 as disclosed herein. Suitable sources of mRNA can be identified by probing northern blots with probes designed from the sequences disclosed herein. A library is then prepared from mRNA of a positive tissue or cell line.

A zacrp7-encoding cDNA can then be isolated by a variety of methods, such as by probing with a complete or partial human cDNA or with one or more sets of degenerate probes based on the disclosed sequences. A cDNA can also be cloned using the polymerase chain reaction with primers designed from the representative human zacrp7 sequences disclosed herein. Within an additional method, the cDNA library can be used to transform or transfect host cells, and expression of the cDNA of interest can be detected with an antibody to zacrp7 polypeptide. Similar techniques can also be applied to the isolation of genomic clones.

Those skilled in the art will recognize that the sequence disclosed in SEQ ID NO:1 represents a single allele of human zacrp7, and that allelic variation and alternative splicing are expected to occur. Allelic variants of this sequence can be cloned by probing cDNA or genomic libraries from different individuals according to standard procedures. Allelic variants of the nucleotide sequence shown in SEQ ID NO:1, including those containing silent mutations and those in which mutations result in amino acid sequence changes, are within the scope of the present invention, as are proteins which are allelic variants of SEQ ID NO:2. cDNA molecules generated from alternatively spliced mRNAs, which retain the properties of the zacrp7 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Within preferred embodiments of the invention, the isolated nucleic acid molecules can hybridize under stringent conditions to nucleic acid molecules having the nucleotide sequence of SEQ ID NO:1 or to nucleic acid molecules having a nucleotide sequence complementary to SEQ ID NO:1. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software, such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below. This allows for the maximum rate of hybridization for DNA-DNA and DNA-RNA hybrids.

The length of the polynucleotide sequence influences the rate and stability of hybrid formation. Smaller probe sequences, <50 base pairs, reach equilibrium with complementary sequences rapidly, but may form less stable hybrids. Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing sodium chloride. Therefore, the higher the G-C content, the more stable the hybrid. Even distribution of G and C residues within the sequence also contribute positively to hybrid stability. In addition, the base pair composition can be manipulated to alter the Tₘ of a given sequence. For example, 5-methyldeoxycytidine can be substituted for deoxycytidine and 5-bromodeoxuridine can be substituted for thymidine to increase the Tₘ, whereas 7-deazz-2′-deoxyguanosine can be substituted for guanosine to reduce dependence on Tₘ.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). By decreasing the ionic concentration of the buffer, the stringency of the hybridization is increased. Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As an illustration, a nucleic acid molecule encoding a variant zacrp7 polypeptide can be hybridized with a nucleic acid molecule having the nucleotide sequence of SEQ ID NO:1 (or its complement) at 42° C. overnight in a solution comprising 50% formamide, 5×SSC (1×SSC: 0.15 M sodium chloride and 15 mM sodium citrate), 50 mM sodium phosphate (pH 7.6), 5× Denhardt's solution (100× Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher or lower temperature, such as about 65° C., in a solution that does not contain formamide. Moreover, premixed hybridization solutions are available (e.g., EXPRESSHYB Hybridization Solution from CLONTECH Laboratories, Inc.), and hybridization can be performed according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant zacrp7 polypeptide hybridize with a nucleic acid molecule having the nucleotide sequence of SEQ ID NO:1 (or its complement) under stringent washing conditions, in which the wash stringency is equivalent to 0.5×-2×SSC with 0.1% SDS at 50-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2×SSC with 0.1% sodium dodecyl sulfate (SDS) at 50-65° C. In other words, nucleic acid molecules encoding a variant zacrp7 polypeptide hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

The present invention also provides isolated zacrp7 polypeptides that have a substantially similar sequence identity to the polypeptides of SEQ ID NO:2, or their orthologs. The term “substantially similar sequence identity” is used herein to denote polypeptides having at least 70%, at least 80%, at least 90%, at least 95% or greater than 95% sequence identity to the sequences shown in SEQ ID NO:2, or their orthologs. The present invention also includes polypeptides that comprise an amino acid sequence having at least 70%, at least 80%, at least 90%, at least 95% or greater than 95% sequence identity to the sequence of amino acid residues 70-252 of SEQ ID NO:2. The present invention further includes nucleic acid molecules that encode such polypeptides. Methods for determining percent identity are described below.

The present invention also contemplates zacrp7 variant nucleic acid molecules that can be identified using two criteria: a determination of the similarity between the encoded polypeptide with the amino acid sequence of SEQ ID NO:2, and a hybridization assay, as described above. Such zacrp7 variants include nucleic acid molecules (1) that hybridize with a nucleic acid molecule having the nucleotide sequence of SEQ ID NO:1 (or its complement) under stringent washing conditions, in which the wash stringency is equivalent to 0.5×-2×SSC with 0.1% SDS at 50-65° C., and (2) that encode a polypeptide having at least 70%, at least 80%, at least 90%, at least 95% or greater than 95% sequence identity to the amino acid sequence of SEQ ID NO:2. Alternatively, zacrp7 variants can be characterized as nucleic acid molecules (1) that hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., and (2) that encode a polypeptide having at least 70%, at least 80%, at least 90%, at least 95% or greater than 95% sequence identity to the amino acid sequence of SEQ ID NO:2.

Percent sequence identity is determined by conventional methods. See, for example, Altschul et al., Bull. Math. Bio. 48:603, 1986, and Henikoff and Henikoff, Proc. Natl. Acad. Sci. USA 89:10915, 1992. Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 4 (amino acids are indicated by the standard one-letter codes). The percent identity is then calculated as: ([Total number of identical matches]/[length of the longer sequence plus the number of gaps introduced into the longer sequence in order to align the two sequences])(100).

TABLE 4

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −4 | −2 | −2 | −4 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zacrp7. The FASTA algorithm is described by Pearson and Lipman, Proc. Natl. Acad. Sci. USA 85:2444, 1988, and by Pearson, Meth. Enzymol. 183:63, 1990.

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then re-scored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63, 1990.

FASTA can also be used to determine the sequence identity of nucleic acid molecules using a ratio as disclosed above. For nucleotide sequence comparisons, the ktup value can range between one to six, preferably from four to six.

The present invention includes nucleic acid molecules that encode a polypeptide having one or more “conservative amino acid substitutions,” compared with the amino acid sequence of SEQ ID NO:2. Conservative amino acid substitutions can be based upon the chemical properties of the amino acids. That is, variants can be obtained that contain one or more amino acid substitutions of SEQ ID NO:2, in which an alkyl amino acid is substituted for an alkyl amino acid in a zacrp7 amino acid sequence, an aromatic amino acid is substituted for an aromatic amino acid in a zacrp7 amino acid sequence, a sulfur-containing amino acid is substituted for a sulfur-containing amino acid in a zacrp7 amino acid sequence, a hydroxy-containing amino acid is substituted for a hydroxy-containing amino acid in a zacrp7 amino acid sequence, an acidic amino acid is substituted for an acidic amino acid in a zacrp7 amino acid sequence, a basic amino acid is substituted for a basic amino acid in a zacrp7 amino acid sequence, or a dibasic monocarboxylic amino acid is substituted for a dibasic monocarboxylic amino acid in a zacrp7 amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

The BLOSUM62 table is an amino acid substitution matrix derived from about 2,000 local multiple alignments of protein sequence segments, representing highly conserved regions of more than 500 groups of related proteins (Henikoff and Henikoff, Proc. Natl. Acad. Sci. USA 89:10915, 1992). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Conservative amino acid changes in a zacrp7 gene can be introduced by substituting nucleotides for the nucleotides recited in SEQ ID NO:1. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)). The ability of such variants to modulate cellular and extracellular interactions or other properties of the wild-type protein as described herein, can be determined using a standard methods, such as the assays described herein. Alternatively, a variant zacrp7 polypeptide can be identified by the ability to specifically bind anti-zacrp7 antibodies.

The proteins of the present invention can also comprise non-naturally occurring amino acid residues. Non-naturally occurring amino acids include, without limitation, trans-3-methylproline, 2,4-methanoproline, cis-4-hydroxy-proline, trans-4-hydroxyproline, N-methylglycine, allo-threonine, methylthreonine, hydroxyethyl-cysteine, hydroxy-ethylhomocysteine, nitroglutamine, homo-glutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722, 1991, Ellman et al., Methods Enzymol. 202:301, 1991, Chung et al., Science 259:806, 1993, and Chung et al., Proc. Nat. Acad. Sci. USA 90:10145, 1993.

In a second method, translation is carried out in Xenopus oocytes by microinjection of mutated mRNA and chemically aminoacylated suppressor tRNAs (Turcatti et al., J. Biol. Chem. 271:19991, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395, 1993).

A limited number of non-conservative amino acids, amino acids that are not encoded by the genetic code, non-naturally occurring amino acids, and unnatural amino acids may be substituted for zacrp7 amino acid residues.

Multiple amino acid substitutions can be made and tested using known methods of mutagenesis and screening, such as those disclosed by Reidhaar-Olson and Sauer (Science 241:53, 1988) or Bowie and Sauer (Proc. Nat. Acad. Sci. USA 86:2152, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832, 1991, Ladner et al., U.S. Pat. No. 5,223,409, Huse, international publication No. WO 92/06204, and region-directed mutagenesis (Derbyshire et al., Gene 46:145, 1986, and Ner et al., DNA 7:127, 1988).

Variants of the disclosed zacrp7 nucleotide and polypeptide sequences can also be generated through DNA shuffling as disclosed by Stemmer, Nature 370:389, 1994, Stemmer, Proc. Nat. Acad. Sci. USA 91:10747, 1994, and international publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind with anti-zacrp7 antibodies, can be recovered from the host cells and rapidly sequenced using modern equipment. These methods allow the rapid determination of the importance of individual amino acid residues in a polypeptide of interest, and can be applied to polypeptides of unknown structure.

Essential amino acids in the polypeptides of the present invention can be identified according to procedures known in the art, such as site-directed mutagenesis or alanine-scanning mutagenesis (Cunningham and Wells, Science 244:1081, 1989, Bass et al., Proc. Nat. Acad. Sci. USA 88:4498, 1991, Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699, 1996. The identities of essential amino acids can also be inferred from analysis of homologies with zacrp7.

The location of zacrp7 receptor binding domains can be identified by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306, 1992, Smith et al., J. Mol. Biol. 224:899, 1992, and Wlodaver et al., FEBS Lett. 309:59, 1992. Moreover, zacrp7 labeled with biotin or FITC can be used for expression cloning of zacrp7 receptors.

The present invention also provides polypeptide fragments or peptides comprising an epitope-bearing portion of a zacrp7 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat. Acad. Sci. USA 81:3998, 1983).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660, 1983). Accordingly, antigenic epitope-bearing peptides and polypeptides of the present invention are useful to raise antibodies that bind with the polypeptides described herein.

Antigenic epitope-bearing peptides and polypeptides preferably contain at least four to ten amino acids, at least ten to fifteen amino acids, or about 15 to about 30 amino acids of SEQ ID NO:2. Such epitope-bearing peptides and polypeptides can be produced by fragmenting a zacrp7 polypeptide, or by chemical peptide synthesis, as described herein. Moreover, epitopes can be selected by phage display of random peptide libraries (see, for example, Lane and Stephen, Curr. Opin. Immunol. 5:268, 1993, and Cortese et al., Curr. Opin. Biotechnol. 7:616, 1996). Standard methods for identifying epitopes and producing antibodies from small peptides that comprise an epitope are described, for example, by Mole, “Epitope Mapping,” in Methods in Molecular Biology, Vol. 10, Manson (ed.), pages 105-16 (The Humana Press, Inc. 1992), Price, “Production and Characterization of Synthetic Peptide-Derived Antibodies, [symbol] in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

Regardless of the particular nucleotide sequence of a variant zacrp7 gene, the gene encodes a polypeptide that is characterized by its ability to modulate cellular or extracellular interactions, or other activities of the wild-type protein as described herein, or by the ability to bind specifically to an anti-zacrp7 antibody. More specifically, variant zacrp7 genes encode polypeptides which exhibit at least 50%, and preferably, greater than 70, 80, or 90%, of the activity of polypeptide encoded by the human zacrp7 gene described herein.

For any zacrp7 polypeptide, including variants and fusion proteins, one of ordinary skill in the art can readily generate a fully degenerate polynucleotide sequence encoding that variant using the information set forth in Tables 2 and 3 above. Moreover, those of skill in the art can use standard software to devise zacrp7 variants based upon the nucleotide and amino acid sequences described herein. Accordingly, the present invention includes a computer-readable medium encoded with a data structure that provides at least one of the following sequences: SEQ ID NO:1, SEQ ID NO:2, and SEQ ID NO:11. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

The present invention further provides a variety of polypeptide fusions and related multimeric proteins comprising one or more polypeptide fusions. For example, a zacrp7 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zacrp7 polypeptide fusions can be expressed in genetically engineered cells to produce a variety of multimeric zacrp7 analogs. Auxiliary domains can be fused to zacrp7 polypeptides to target them to specific cells, tissues, or macromolecules (e.g., collagen). For example, a zacrp7 polypeptide or protein could be targeted to a predetermined cell type by fusing a zacrp7 polypeptide to a ligand that specifically binds to a receptor on the surface of the target cell. In this way, polypeptides and proteins can be targeted for therapeutic or diagnostic purposes. A zacrp7 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

Zacrp7 fusion proteins of the present invention encompass (1) a polypeptide selected from the group consisting of: (a) polypeptide molecules comprising a sequence of amino acid residues as shown in SEQ ID NO:2 from amino acid residue 1 (Met), 31 (Gln) or 51 (Gly) to amino acid residue 303 (Leu); (b) polypeptide molecules ranging from amino acid 51 (Gly) to amino acid 153 (Cys) of SEQ ID NO:2, a portion of the zacrp7 polypeptide containing the collagen-like domain or a portion of the collagen-like domain capable of dimerization or oligomerization; (c) polypeptide molecules ranging from amino acid 154 (Arg) to 303 (Leu) of SEQ ID NO:2, a portion of the zacrp7 polypeptide containing the C1q domain or an active portion of the C1q domain; or (d) polypeptide molecules ranging from amino acid 51 (Gly) to 303 (Leu), a portion of the zacrp7 polypeptide including the collagen-like domain and the Clq domain; and (2) another polypeptide. The other polypeptide may be alternative or additional C1q domain, an alternative or additional collagen-like domain, a signal peptide to facilitate secretion of the fusion protein or the like. Such domains can be obtained from other adipocyte complement related protein family members, other proteins having collagen and/or C1q domains as disclosed herein. The globular domain of complement binds IgG, thus, the globular domain of zacrp7 polypeptide, fragment or fusion may have a similar role.

Zacrp7 polypeptides, ranging from amino acid 1 (Met) to amino acid 303 (Leu); the mature zacrp7 polypeptides, ranging from amino acid 31 (Gln) to amino acid 303 (Leu); or the secretion leader fragments thereof, which fragments range from amino acid 1 (Met) to amino acid 30 (Gly) may be used in the study of secretion of proteins from cells. In preferred embodiments of this aspect of the present invention, the mature polypeptides are formed as fusion proteins with putative secretory signal sequences; plasmids bearing regulatory regions capable of directing the expression of the fusion protein is introduced into test cells; and secretion of mature protein is monitored. The monitoring may be done by techniques known in the art, such as HPLC and the like.

The polypeptides of the present invention, including full-length proteins, fragments thereof and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., ibid., and Ausubel et al. ibid.

In general, a DNA sequence encoding a zacrp7 polypeptide of the present invention is operably linked to other genetic elements required for its expression, generally including a transcription promoter and terminator within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zacrp7 polypeptide into the secretory pathway of a host cell, a secretory signal sequence (also known as a leader sequence, signal sequence, prepro sequence or pre-sequence) is provided in the expression vector. The secretory signal sequence may be that of the zacrp7 polypeptide, or may be derived from another secreted protein (e.g., t-PA) or synthesized de novo. The secretory signal sequence is joined to the zacrp7 polypeptide DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). Conversely, the signal sequence portion of the zacrp7 polypeptide (amino acid residues 1-30 of SEQ ID NO:2) may be employed to direct the secretion of an alternative protein by analogous methods.

The secretory signal sequence contained in the polypeptides of the present invention can be used to direct other polypeptides into the secretory pathway. The present invention provides for such fusion polypeptides. A signal fusion polypeptide can be made wherein a secretory signal sequence derived from amino acid residues 1-30 of SEQ ID NO:2 is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein, such as a receptor. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6; 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61, DG44 CHO, Chasin et al., Som. Cell. Molec. Genet. 12:555-666, 1986) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant zacrp7 in baculovirus utilizes a transposon-based system described by Luckow (Luckow et al., J. Virol. 67:4566-79, 1993). This system, which utilizes transfer vectors, is sold in the Bac-to-Bac™ kit (Life Technologies, Rockville, Md.). This system utilizes a transfer vector, pFastBacl™ (Life Technologies) containing a Tn7 transposon to move the DNA encoding the zacrp7 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zacrp7. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins and Possee, J. Gen. Virol. 71:971-6, 1990; Bonning et al., J. Gen. Virol. 75:1551-6, 1994; and, Chazenbalk, and Rapoport, J. Biol. Chem. 270:1543-9, 1995. In such transfer vector constructs, a short or long version of the basic protein promoter can be used. Moreover, transfer vectors can be constructed which replace the native zacrp7 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zacrp7 secretory signal sequence. In addition, transfer vectors can include an in-frame fusion with DNA encoding an epitope tag at the C- or N-terminus of the expressed zacrp7 polypeptide, for example, a Glu-Glu epitope tag (Grussenmeyer et al., Proc. Natl. Acad. Sci. 82:7952-4, 1985) Using a technique known in the art, a transfer vector containing zacrp7 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zacrp7 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King and Possee, ibid.; O'Reilly et al., ibid.; Richardson, ibid.). Subsequent purification of the zacrp7 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (T) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zacrp7 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis, against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Expressed recombinant zacrp7 polypeptides (or zacrp7 fragments or fusion polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their structural or binding properties. For example, immobilized metal ion adsorption (IMAC) chromatography can be used to purify histidine-rich proteins or proteins having a His tag. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, Deutscher, (ed.), Acad. Press, San Diego, 1990, pp. 529-39). Within an additional preferred embodiments of the invention, a fusion of the polypeptide of interest and an affinity tag (e.g., maltose-binding protein, FLAG, Glu-Glu, an immunoglobulin domain) may be constructed to facilitate purification as is discussed in greater detail in the Example sections below.

Protein refolding (and optionally, reoxidation) procedures may be advantageously used. It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

Zacrp7 polypeptides or fragments thereof may also be prepared through chemical synthesis by methods well known in the art, such as exclusive solid phase synthesis, partial solid phase methods, fragment condensation or classical solution synthesis, see for example, Merrifield, J. Am. Chem. Soc. 85:2149, 1963. Such zacrp7 polypeptides may be monomers or multimers; glycosylated or non-glycosylated; pegylated or non-pegylated; and may or may not include an initial methionine amino acid residue.

A ligand-binding polypeptide, such as a zacrp7-binding polypeptide, can also be used for purification of ligand. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the ligand-binding polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/anti-complement pair) or a binding fragment thereof, and a commercially available biosensor instrument (BIAcore™, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

The invention also provides anti-zacrp7 antibodies. Antibodies to zacrp7 can be obtained, for example, using as an antigen the product of a zacrp7 expression vector, or zacrp7 isolated from a natural source. Particularly useful anti-zacrp7 antibodies “bind specifically” with zacrp7. Antibodies are considered to be specifically binding if the antibodies bind to a zacrp7 polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660, 1949). Suitable antibodies include antibodies that bind with zacrp7 in particular domains.

Anti-zacrp7 antibodies can be produced using antigenic zacrp7 epitope-bearing peptides and polypeptides. Antigenic epitope-bearing peptides and polypeptides of the present invention contain a sequence of at least nine, preferably between 15 to about 30 amino acids contained within SEQ ID NO:2. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with zacrp7. It is desirable that the amino acid sequence of the epitope-bearing peptide is selected to provide substantial solubility in aqueous solvents (i.e., the sequence includes relatively hydrophilic residues, while hydrophobic residues are preferably avoided). Hydrophilic peptides can be predicted by one of skill in the art from a hydrophobicity plot, see for example, Hopp and Woods (Proc. Nat. Acad. Sci. USA 78:3824-8, 1981) and Kyte and Doolittle (J. Mol. Biol. 157: 105-142, 1982). Moreover, amino acid sequences containing proline residues may be also be desirable for antibody production.

Polyclonal antibodies to recombinant zacrp7 protein or to zacrp7 isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a zacrp7 polypeptide can be increased through the use of an adjuvant, such as alum (aluminum hydroxide) or Freund's complete or incomplete adjuvant. Polypeptides useful for immunization also include fusion polypeptides, such as fusions of zacrp7 or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, hamsters, guinea pigs, goats, or sheep, an anti-zacrp7 antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310, 1990. Antibodies can also be raised in transgenic animals such as transgenic sheep, cows, goats or pigs, and can also be expressed in yeast and fungi in modified forms as will as in mammalian and insect cells.

Alternatively, monoclonal anti-zacrp7 antibodies can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1-2.6.7 (John Wiley & Sons 1991), Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a zacrp7 gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-zacrp7 antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13, 1994, Lonberg et al., Nature 368:856, 1994, and Taylor et al., Int. Immun. 6:579, 1994.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-zacrp7 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230, 1960, Porter, Biochem. J. 73:119, 1959, Edelman et al., in Methods in Enzymology Vol. 1, page 422 (Academic Press 1967), and by Coligan, ibid.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Natl. Acad. Sci. USA 69:2659, 1972. Alternatively, the variable chains can be linked by an intermolecular disulfide bond or cross-linked by chemicals such as gluteraldehyde (see, for example, Sandhu, Crit. Rev. Biotech. 12:437, 1992).

The Fv fragments may comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97, 1991, also see, Bird et al., Science 242:423, 1988, Ladner et al., U.S. Pat. No. 4,946,778, Pack et al., Bio/Technology 11:1271, 1993, and Sandhu, ibid.

As an illustration, a scFV can be obtained by exposing lymphocytes to zacrp7 polypeptide in vitro, and selecting antibody display libraries in phage or similar vectors (for instance, through use of immobilized or labeled zacrp7 protein or peptide). Genes encoding polypeptides having potential zacrp7 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the zacrp7 sequences disclosed herein to identify proteins which bind to zacrp7.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106, 1991); Courtenay-Luck, “Genetic Manipulation of Monoclonal Antibodies,” in Monoclonal Antibodies: Production, Engineering and Clinical Application, Ritter et al. (eds.), page 166 (Cambridge University Press, 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-zacrp7 antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat. Acad. Sci. USA 86:3833, 1989. Techniques for producing humanized monoclonal antibodies are described, for example, by Jones et al., Nature 321:522, 1986, Carter et al., Proc. Nat. Acad. Sci. USA 89:4285, 1992, Sandhu, Crit. Rev. Biotech. 12:437, 1992, Singer et al., J. Immun. 150:2844, 1993, Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399-434 (John Wiley & Sons, Inc. 1996), and by Queen et al., U.S. Pat. No. 5,693,762 (1997).

Polyclonal anti-idiotype antibodies can be prepared by immunizing animals with anti-zacrp7 antibodies or antibody fragments, using standard techniques. See, for example, Green et al., “Production of Polyclonal Antisera,” in Methods In Molecular Biology: Immunochemical Protocols, Manson (ed.), pages 1-12 (Humana Press 1992). Also, see Coligan, ibid. at pages 2.4.1-2.4.7. Alternatively, monoclonal anti-idiotype antibodies can be prepared using anti-zacrp7 antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875, 1996.

Genes encoding polypeptides having potential zacrp7 polypeptide binding domains, “binding proteins”, can be obtained by screening random or directed peptide libraries displayed on phage (phage display) or on bacteria, such as E. coli. Nucleotide sequences encoding the polypeptides can be obtained in a number of ways, such as through random mutagenesis and random polynucleotide synthesis. Alternatively, constrained phage display libraries can also be produced. These peptide display libraries can be used to screen for peptides which interact with a known target which can be a protein or polypeptide, such as a ligand or receptor, a biological or synthetic macromolecule, or organic or inorganic substances. Techniques for creating and screening such peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Peptide display libraries can be screened using the zacrp7 sequences disclosed herein to identify proteins which bind to zacrp7. These “binding proteins” which interact with zacrp7 polypeptides can be used essentially like an antibody.

A variety of assays known to those skilled in the art can be utilized to detect antibodies and/or binding proteins which specifically bind to zacrp7 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zacrp7 protein or polypeptide.

Antibodies and binding proteins to zacrp7 may be used for tagging cells that express zacrp7; for isolating zacrp7 by affinity purification; for diagnostic assays for determining circulating levels of zacrp7 polypeptides; for detecting or quantitating soluble zacrp7 as marker of underlying pathology or disease; in analytical methods employing FACS; for screening expression libraries; for generating anti-idiotypic antibodies; and as neutralizing antibodies or as antagonists to block zacrp7 polypeptide modulation of spermatogenesis or like activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Moreover, antibodies to zacrp7 or fragments thereof may be used in vitro to detect denatured zacrp7 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zacrp7 polypeptides or anti-zacrp7 antibodies, or bioactive fragments or portions thereof, can be coupled to detectable or cytotoxic molecules and delivered to a mammal having cells, tissues or organs that express the anti-complementary molecule.

An additional aspect of the present invention provides methods for identifying agonists or antagonists of the zacrp7 polypeptides disclosed above, which agonists or antagonists may have valuable properties as discussed further herein. Within one embodiment, there is provided a method of identifying zacrp7 polypeptide agonists, comprising providing cells responsive thereto, culturing the cells in the presence of a test compound and comparing the cellular response with the cell cultured in the presence of the zacrp7 polypeptide, and selecting the test compounds for which the cellular response is of the same type.

Within another embodiment, there is provided a method of identifying antagonists of zacrp7 polypeptide, comprising providing cells responsive to a zacrp7 polypeptide, culturing a first portion of the cells in the presence of zacrp7 polypeptide, culturing a second portion of the cells in the presence of the zacrp7 polypeptide and a test compound, and detecting a decrease in a cellular response of the second portion of the cells as compared to the first portion of the cells. In addition to those assays disclosed herein, samples can be tested for inhibition of zacrp7 activity within a variety of assays designed to measure receptor binding or the stimulation/inhibition of zacrp7-dependent cellular responses. For example, zacrp7-responsive cell lines can be transfected with a reporter gene construct that is responsive to a zacrp7-stimulated cellular pathway. Reporter gene constructs of this type are known in the art, and will generally comprise a zacrp7-DNA response element operably linked to a gene encoding an assayable protein, such as luciferase. DNA response elements can include, but are not limited to, cyclic AMP response elements (CRE), hormone response elements (HRE), insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6, 1988 and Habener, Molec. Endocrinol. 4 (8):1087-94, 1990. Hormone response elements are reviewed in Beato, Cell 56:335-44; 1989. Candidate compounds, solutions, mixtures or extracts are tested for the ability to inhibit the activity of zacrp7 on the target cells as evidenced by a decrease in zacrp7 stimulation of reporter gene expression. Assays of this type will detect compounds that directly block zacrp7 binding to cell-surface receptors, as well as compounds that block processes in the cellular pathway subsequent to receptor-ligand binding. In the alternative, compounds or other samples can be tested for direct blocking of zacrp7 binding to receptor using zacrp7 tagged with a detectable label (e.g., ¹²⁵I, biotin, horseradish peroxidase, FITC, and the like). Within assays of this type, the ability of a test sample to inhibit the binding of labeled zacrp7 to the receptor is indicative of inhibitory activity, which can be confirmed through secondary assays. Receptors used within binding assays may be cellular receptors or isolated, immobilized receptors.

Adipocyte complement related proteins are involved in cell-cell or cell-extracellular matrix interactions, particularly those involving modulation of tissue remodeling. The phenotypic manifestation of many autoimmune and remodeling-related diseases is extensive activation of inflammatory and/or tissue remodeling processes. The result is often that functional organ or sub-organ tissue is replaced by a variety of extracellular matrix (ECM) components incapable of performing the function of the replaced biological structure. There is an incomplete understanding of the initiation events in these diseases, and the resulting excessive extracellular matrix deposition. The initiation events have been hypothesized to involve an injury or initial perturbation of the optimal biological structure regulation. Interestingly, sometimes intracellular components are found as autoantigens, indicative of particular diseases. It could be that the production of antibodies by the immune system, after excessive exposure to these intracellular proteins, is a result of excessive or improper remodeling. By targeting the remodeling process it may be possible to lessen the effect autoantigens. Therefore, zacrp7 polypeptides, fragments, fusions, agonists, antagonists and the like-would be beneficial in mediating a variety of autoimmune and remodeling diseases.

It is possible that an improper remodeling response to connective tissue or muscle injury in the joints results in sensitivity to excessive release of cellular components at the site of the injury. Zacrp6 polypeptides, fragments, fusions and the like would be useful in determining if an association exists between such a response and the inflammation associated with arthritis. Such indicators include a reduction in inflammation and relief of pain or stiffness. In animal models, indications would be derived from macroscopic inspection of joints and change in swelling of hind paws. Zacrp6 polypeptides, fragments, fusions and the like can be administered to animal models of osteoarthritis (Kikuchi et al., Osteoarthritis Cartilage 6:177-86, 1998 and Lohmander et al., Arthritis Rheum. 42:534-44, 1999) to look for inhibition of tissue destruction that results from inflammation stimulated by the action of collagenase.

Recent findings have shown that autoantigens diagnostic of scleroderma are to what would be consider cytoplasmic proteins. A knockout animal for Zacrp6, as described herein, would be useful in determining if antibodies to zacrp6 proteins, fragments, fusions and the like are raised as a response to inflammation due to improper or incomplete repair of local tissue in response to stress.

Zacrp7 polypeptides, fragments, fusions and the like, as provided herein, would be useful in determining if excessive and/or inappropriate arterial remodeling plays a role in plaque formation in arterial sclerosis and arterial injury, such as arterial occlusion, using methods provided herein. Treatment of a vascular injury (and underlying extracellular matrix) with adipocyte complement protein zsig37 appears to alter the process of vascular remodeling at a very early stage (co-pending U.S. patent Ser. No. 09/253,604). Treatment with an adipocyte complement protein may act to keep platelets relatively quiescent after injury, eliminating excessive recruitment of pro-remodeling and proinflammatory proteins and cells.

Other members of the family may modulate remodeling induced by the presence of fat, or cholesterol for instance. Excessive amounts of cholesterol and fat in the blood might activate remodeling, in the absence of the correct adipocyte complement protein family member.

ACRP30 is expressed only in actively proliferating adipose tissue. Connective tissue remodeling is tightly linked to this activation of fat cells. There is clearly a link between excessive weight gain (fat) and diabetes. It is therefore likely that ACRP30 is involved in fat remodeling and this process is overtaxed in obese individuals. As a result, the effects of improper and inadequate fat storage contribute to the onset of Type II diabetes.

Energy balance (involving energy metabolism, nutritional state, lipid storage and the like) is an important criteria for health. This energy homeostasis involves food intake and metabolism of carbohydrates and lipids to generate energy necessary for voluntary and involuntary functions. Metabolism of proteins can lead to energy generation, but preferably leads to muscle formation or repair. Among other consequences, a lack of energy homeostasis lead to over or under formation of adipose tissue. Formation and storage of fat is insulin-modulated. For example, insulin stimulates the transport of glucose into cells, where it is metabolized into α-glycerophosphate which is used in the esterification of fatty acids to permit storage thereof as triglycerides. In addition, adipocytes (fat cells) express a specific transport protein that enhances the transfer of free fatty acids into adipocytes.

Adipocytes also secrete several proteins believed to modulate homeostatic control of glucose and lipid metabolism. These additional adipocyte-secreted proteins include adipsin, complement factors C3 and B, tumor necrosis factor α, the ob gene product and Acrp30. Evidence also exists suggesting the existence of an insulin-regulated secretory pathway in adipocytes. Scherer et al., J. Biol. Chem. 270(45): 26746-9, 1995. Over or under secretion of these moieties, impacted in part by over or under formation of adipose tissue, can lead to pathological conditions associated directly or indirectly with obesity or anorexia.

Based on homology to other adipocyte complement related proteins, such as ACRP30, zacrp7 polypeptides, fragments, fusions, agonists or antagonists can be used to modulate energy balance in mammals or to protect endothelial cells from injury. With regard to modulating energy balance, zacrp7 polypeptides modulate cellular metabolic reactions. Such metabolic reactions include adipogenesis, gluconeogenesis, glycogenolysis, lipogenesis, glucose uptake, protein synthesis, thermogenesis, oxygen utilization and the like. Zacrp7 polypeptides may also find use as neurotransmitters or as modulators of neurotransmission, as indicated by expression of the polypeptide in tissues associated with the sympathetic or parasympathetic nervous system. In this regard, zacrp7 polypeptides may find utility in modulating nutrient uptake, as demonstrated, for example, by 2-deoxy-glucose uptake in the brain or the like.

Among other methods known in the art or described herein, mammalian energy balance may be evaluated by monitoring one or more of the following metabolic functions: adipogenesis, gluconeogenesis, glycogenolysis, lipogenesis, glucose uptake, protein synthesis, thermogenesis, oxygen utilization or the like. These metabolic functions are monitored by techniques (assays or animal models) known to one of ordinary skill in the art, as is more fully set forth below. For example, the glucoregulatory effects of insulin are predominantly exerted in the liver, skeletal muscle and adipose tissue. Insulin binds to its cellular receptor in these three tissues and initiates tissue-specific actions that result in, for example, the inhibition of glucose production and the stimulation of glucose utilization. In the liver, insulin stimulates glucose uptake and inhibits gluconeogenesis and glycogenolysis. In skeletal muscle and adipose tissue, insulin acts to stimulate the uptake, storage and utilization of glucose.

Art-recognized methods exist for monitoring all of the metabolic functions recited above. Thus, one of ordinary skill in the art is able to evaluate zacrp7 polypeptides, fragments, fusion proteins, antibodies, agonists and antagonists for metabolic modulating functions. Exemplary modulating techniques are set forth below.

Adipogenesis, gluconeogenesis and glycogenolysis are interrelated components of mammalian energy balance, which may be evaluated by known techniques using, for example, ob/ob mice or db/db mice. The ob/ob mice are inbred mice that are homozygous for an inactivating mutation at the ob (obese) locus. Such ob/ob mice are hyperphagic and hypometabolic, and are believed to be deficient in production of circulating OB protein. The db/db mice are inbred mice that are homozygous for an inactivating mutation at the db (diabetes) locus. The db/db mice display a phenotype similar to that of ob/ob mice, except db/db mice also display a diabetic phenotype. Such db/db mice are believed to be resistant to the effects of circulating OB protein. Also, various in vitro methods of assessing these parameters are known in the art.

Insulin-stimulated lipogenesis, for example, may be monitored by measuring the incorporation of ¹⁴C-acetate into triglyceride (Mackall et al. J. Biol. Chem. 251:6462-4, 1976) or triglyceride accumulation (Kletzien et al., Mol. Pharmacol. 41:393-8, 1992).

Glucose uptake may be evaluated, for example, in an assay for insulin-stimulated glucose transport. Non-transfected, differentiated L6 myotubes (maintained in the absence of G418) are placed in DMEM containing 1 g/l glucose, 0.5 or 1.0% BSA, 20 mM Hepes, and 2 mM glutamine. After two to five hours of culture, the medium is replaced with fresh, glucose-free DMEM containing 0.5 or 1.0% BSA, 20 mM Hepes, 1 mM pyruvate, and 2 mM glutamine. Appropriate concentrations of insulin or IGF-1, or a dilution series of the test substance, are added, and the cells are incubated for 20-30 minutes. ³H or ¹⁴C-labeled deoxyglucose is added to ≈50 ┐M final concentration, and the cells are incubated for approximately 10-30 minutes. The cells are then quickly rinsed with cold buffer (e.g. PBS), then lysed with a suitable lysing agent (e.g. 1% SDS or 1 N NaOH). The cell lysate is then evaluated by counting in a scintillation counter. Cell-associated radioactivity is taken as a measure of glucose transport after subtracting non-specific binding as determined by incubating cells in the presence of cytocholasin b, an inhibitor of glucose transport. Other methods include those described by, for example, Manchester et al., Am. J. Physiol. 266 (Endocrinol. Metab. 29):E326-E333, 1994 (insulin-stimulated glucose transport).

Protein synthesis may be evaluated, for example, by comparing precipitation of ³⁵S-methionine-labeled proteins following incubation of the test cells with ³⁵S-methionine and ³⁵S-methionine and a putative modulator of protein synthesis.

Thermogenesis may be evaluated as described by B. Stanley in The Biology of Neuropeptide Y and Related Peptides, W. Colmers and C. Wahlestedt (eds.), Humana Press, Ottawa, 1993, pp. 457-509; C. Billington et al., Am. J. Physiol. 260:R321, 1991; N. Zarjevski et al., Endocrinology 133:1753, 1993; C. Billington et al., Am. J. Physiol. 266:R1765, 1994; Heller et al., Am. J. Physiol. 252(4 Pt 2): R661-7, 1987; and Heller et al., Am. J. Physiol. 245: R321-8, 1983. Also, metabolic rate, which may be measured by a variety of techniques, is an indirect measurement of thermogenesis.

Oxygen utilization may be evaluated as described by Heller et al., Pflugers Arch 369: 55-9, 1977. This method also involved an analysis of hypothalmic temperature and metabolic heat production. Oxygen utilization and thermoregulation have also been evaluated in humans as described by Haskell et al., J. Appl. Physiol. 51: 948-54, 1981.

Neurotransmission functions may be evaluated by monitoring 2-deoxy-glucose uptake in the brain. This parameter is monitored by techniques (assays or animal models) known to one of ordinary skill in the art, for example, autoradiography. Useful monitoring techniques are described, for example, by Kilduff et al., J. Neurosci. 10 2463-75, 1990, with related techniques used to evaluate the “hibernating heart” as described in Gerber et al. Circulation 94: 651-8, 1996, and Fallavollita et al., Circulation 95: 1900-9, 1997.

In addition, zacrp7 polypeptides, fragments, fusions agonists or antagonists thereof may be therapeutically useful for anti-microbial applications. For example, complement component C1q plays a role in host defense against infectious agents, such as bacteria and viruses. C1q is known to exhibit several specialized functions. For example, C1q triggers the complement cascade via interaction with bound antibody or C-reactive protein (CRP). Also, Clq interacts directly with certain bacteria, RNA viruses, mycoplasma, uric acid crystals, the lipid A component of bacterial endotoxin and membranes of certain intracellular organelles. C1q binding to the C1q receptor is believed to promote phagocytosis. C1q also appears to enhance the antibody formation aspect of the host defense system. See, for example, Johnston, Pediatr. Infect. Dis. J. 12(11): 933-41, 1993. Thus, soluble C1q-like molecules may be useful as anti-microbial agents, promoting lysis or phagocytosis of infectious agents.

Zacrp7 fragments as well as zacrp7 polypeptides, fusion proteins, agonists, antagonists or antibodies may be evaluated with respect to their anti-microbial properties according to procedures known in the art. See, for example, Barsum et al., Eur. Respir. J. 8 (5): 709-14, 1995; Sandovsky-Losica et al., J. Med. Vet. Mycol (England) 28 (4): 279-87, 1990; Mehentee et al., J. Gen. Microbiol. (England) 135 (Pt. 8): 2181-8, 1989; Segal and Savage, J. Med. Vet. Mycol. 24: 477-9, 1986 and the like. If desired, the performance of zacrp7 in this regard can be compared to proteins known to be functional in this regard, such as proline-rich proteins, lysozyme, histatins, lactoperoxidase or the like. In addition, zacrp7 fragments, polypeptides, fusion proteins, agonists, antagonists or antibodies may be evaluated in combination with one or more anti-microbial agents to identify synergistic effects. One of ordinary skill in the art will recognize that the anti-microbial properties of zacrp7 polypeptides, fragments, fusion proteins, agonists, antagonists and antibodies may be similarly evaluated.

As neurotransmitters or neurotransmission modulators, zacrp7 polypeptide fragments as well as zacrp7 polypeptides, fusion proteins, agonists, antagonists or antibodies of the present invention may also modulate calcium ion concentration, muscle contraction, hormone secretion, DNA synthesis or cell growth, inositol phosphate turnover, arachidonate release, phospholipase-C activation, gastric emptying, human neutrophil activation or ADCC capability, superoxide anion production and the like. Evaluation of these properties can be conducted by known methods, such as those set forth herein.

The impact of zacrp7 polypeptide, fragment, fusion, antibody, agonist or antagonist on intracellular calcium level may be assessed by methods known in the art, such as those described by Dobrzanski et al., Regulatory Peptides 45: 341-52, 1993, and the like. The impact of zacrp7 polypeptide, fragment, fusion, agonist or antagonist on muscle contraction may be assessed by methods known in the art, such as those described by Smits & Lebebvre, J. Auton. Pharmacol. 14: 383-92, 1994, Belloli et al., J. Vet. Pharmacol. Therap. 17: 379-83, 1994, Maggi et al., Regulatory Peptides 53: 259-74, 1994, and the like. The impact of zacrp7 polypeptide, fragment, fusion, agonist or antagonist on hormone secretion may be assessed by methods known in the art, such as those for prolactin release described by Henriksen et al., J. Recep. Sig. Transd. Res. 15(1-4): 529-41, 1995, and the like. The impact of zacrp7 polypeptide, fragment, fusion, agonist or antagonist on DNA synthesis or cell growth may be assessed by methods known in the art, such as those described by Dobrzanski et al., Regulatory Peptides 45: 341-52, 1993, and the like. The impact of zacrp7 polypeptide, fragment, fusion, agonist or antagonist on inositol phosphate turnover may be assessed by methods known in the art, such as those described by Dobrzanski et al., Regulatory Peptides 45: 341-52, 1993, and the like.

Also, the impact of zacrp7 polypeptide, fragment, fusion, agonist or antagonist on arachidonate release may be assessed by methods known in the art, such as those described by Dobrzanski et al., Regulatory Peptides 45: 341-52, 1993, and the like. The impact of zacrp7 polypeptide, fragment, fusion, agonist or antagonist on phospholipase-C activation may be assessed by methods known in the art, such as those described by Dobrzanski et al., Regulatory Peptides 45: 341-52, 1993, and the like. The impact of zacrp7 polypeptide, fragment, fusion, agonist or antagonist on gastric emptying may be assessed by methods known in the art, such as those described by Varga et al., Eur. J. Pharmacol. 286: 109-112,-1995, and the like. The impact of zacrp7 polypeptide, fragment, fusion, agonist or antagonist on human neutrophil activation and ADCC capability may be assessed by methods known in the art, such as those described by Wozniak et al., Immunology 78: 629-34, 1993, and the like. The impact of zacrp7 polypeptide, fragment, fusion, agonist or antagonist on superoxide anion production may be assessed by methods known in the art, such as those described by Wozniak et al., Immunology 78: 629-34, 1993, and the like.

Collagen is a potent inducer of platelet aggregation. This poses risks to patients recovering from vascular injures. Inhibitors of collagen-induced platelet aggregation would be useful for blocking the binding of platelets to collagen-coated surfaces and reducing associated collagen-induced platelet aggregation. C1q is a component of the complement pathway and has been found to stimulate defense mechanisms as well as trigger the generation of toxic oxygen species that can cause tissue damage (Tenner, Behring Inst. Mitt. 93:241-53, 1993). C1q binding sites are found on platelets. Clq, independent of an immune binding partner, has been found to inhibit platelet aggregation but not platelet adhesion or shape change. The amino terminal region of C1q shares homology with collagen (Peerschke and Ghebrehiwet, J. Immunol. 145:2984-88, 1990). Inhibition of C1q and the complement pathway can be determined using methods disclosed herein or know in the art, such as described in Suba and Csako, J. Immunol. 117:304-9, 1976.

The impact of zacrp7 polypeptides, fragments, fusions, agonists or antagonists on complement inhibition may be assessed by methods known in the art. The impact of zacrp7 polypeptide, fragment, fusion, agonist or antagonist on C1q binding activity may be assessed by methods known in the art.

The impact of zacrp7 polypeptide, fragments, fusions, agonists or antagonists on collagen-mediated platelet adhesion, activation and aggregation may be evaluated using methods described herein or known in the art, such as the platelet aggregation assay (Chiang et al., Thrombosis Res. 37:605-12, 1985) and platelet adhesion assays (Peerschke and Ghebrehiwet, J. Immunol. 144:221-25, 1990). Assays for platelet adhesion to collagen and inhibition of collagen-induced platelet aggregation can be measured using methods described in Keller et al., J. Biol. Chem. 268:5450-6, 1993; Waxman and Connolly, J. Biol. Chem. 268:5445-9, 1993; Noeske-Jungblut et al., J. Biol. Chem. 269:5050-3 or 1994 Deckmyn et al., Blood 85:712-9, 1995.

The impact of zacrp7 polypeptide, fragments, fusions, agonists or antagonists on vasodilation of aortic rings can be measured according to the methods of Dainty et al., J. Pharmacol. 100:767, 1990 and Rhee et al., Neurotox. 16:179, 1995.

Various in vitro and in vivo models are available for assessing the effects of zacrp7 polypeptides, fragments, fusion proteins, antibodies, agonists and antagonists on ischemia and reperfusion injury. See for example, Shandelya et al., Circulation 88:2812-26, 1993; Weisman et al., Science 249:146-151, 1991; Buerke et al., Circulation 91:393-402, 1995; Horstick et al., Circulation 95:701-8, 1997 and Burke et al., J. Phar. Exp. Therp. 286:429-38, 1998. An ex vivo hamster platelet aggregation assay is described by Deckmyn et al., ibid. Bleeding times in hamsters and baboons can be measured following injection of zacrp7 polypeptides using the model described by Deckmyn et al., ibid. The formation of thrombus in response to administration of proteins of the present invention can be measured using the hamster femoral vein thrombosis model is provided by Deckmyn et al., ibid. Changes in platelet adhesion under flow conditions following administration of zacrp7 can be measured using the method described in Harsfalvi et al., Blood 85:705-11, 1995.

Complement inhibition and wound healing can be zacrp7 polypeptides, fragments, fusion proteins, antibodies, agonists or antagonists be assayed alone or in combination with other know inhibitors of collagen-induced platelet activation and aggregation, such as palldipin, moubatin or calin, for example.

Zacrp7 polypeptides, fragments, fusion proteins, antibodies, agonists or antagonists can be evaluated using methods described herein or known in the art, such as healing of dermal layers in pigs (Lynch et al., Proc. Natl. Acad. Sci. USA 84: 7696-700, 1987) and full-thickness skin wounds in genetically diabetic mice (Greenhalgh et al., Am. J. Pathol. 136: 1235-46, 1990), for example. The polypeptides of the present invention can be assayed alone or in combination with other known complement inhibitors as described above.

Radiation hybrid mapping is a somatic cell genetic technique developed for constructing high-resolution, contiguous maps of mammalian chromosomes (Cox et al., Science 250:245-50, 1990). Partial or full knowledge of a gene's sequence allows the designing of PCR primers suitable for use with chromosomal radiation hybrid mapping panels. Commercially available radiation hybrid mapping panels which cover the entire human genome, such as the Stanford G3 RH Panel and the GeneBridge 4 RH Panel (Research Genetics, Inc., Huntsville, Ala.), are available. These panels enable rapid, PCR based, chromosomal localizations and ordering of genes, sequence-tagged sites (STSs), and other nonpolymorphic- and polymorphic markers within a region of interest. This includes establishing directly proportional physical distances between newly discovered genes of interest and previously mapped markers. The precise knowledge of a gene's position can be useful in a number of ways including: 1) determining if a sequence is part of an existing contig and obtaining additional surrounding genetic sequences in various forms such as YAC-, BAC- or cDNA clones, 2) providing a possible candidate gene for an inheritable disease which shows linkage to the same chromosomal region, and 3) for cross-referencing model organisms such as mouse which may be beneficial in helping to determine what function a particular gene might have. Radiation hybrid mapping was used to confirm the localization of zacrp7 on human chromosome 4p15. The results showed linkage of zacrp7 to the chromosome 4 marker SHGC-35585 with a LOD score of >16 and at a distance of 0 cR_{—}10000 from the marker. The use of surrounding genes/markers positions zacrp7 in the 4p15 chromosomal region.

Cholecystokinin A receptor (CCKAR) maps to 4p15.2-p15.l. A missense variant, gly21-arg, was found in an African-American with obesity and noninsulin-dependent diabetes (Inoue et al., Genomics 42:331-5, 1997).

CD8, an ecto-nicotinamide adenine dinucleotide glycohydrolase, expressed on hematopoietic cells maps to 4p15. Studies with CD8 knock-out mice indicate that CD8 plays a role in in vivo regulation of humoral immune response (Cockanye et al., Blood 92:1324-33, 1998). CD8 also plays a role in synthesis and hydrolysis of cyclic ADP-ribose in the process of insulin secretion in pancreatic β-cells (Takasawa et al., J. Biol. Chem. 268:26052-4, 1993). CD38 was also identified as an antigen on the cell surface of acute lymphoblastic leukemia (ALL) cells (Katz et al., Europ. J. Immun. 13:1008-13, 1983).

The present invention also provides reagents which will find use in diagnostic applications. For example, the zacrp7 gene, a probe comprising zacrp7 DNA or RNA, or a subsequence thereof can be used to determine if the zacrp7 gene is present on chromosome 4 or if a mutation has occurred. Detectable chromosomal aberrations at the zacrp7 gene locus include, but are not limited to, aneuploidy, gene copy number changes, insertions, deletions, restriction site changes and rearrangements. These aberrations can occur within the coding sequence, within introns, or within flanking sequences, including upstream promoter and regulatory regions, and may be manifested as physical alterations within a coding sequence or changes in gene expression level.

In general, these diagnostic methods comprise the steps of (a) obtaining a genetic sample from a patient; (b) incubating the genetic sample with a polynucleotide probe or primer as disclosed above, under conditions wherein the polynucleotide will hybridize to complementary polynucleotide sequence, to produce a first reaction product; and (iii) comparing the first reaction product to a control reaction product. A difference between the first reaction product and the control reaction product is indicative of a genetic abnormality in the patient. Genetic samples for use within the present invention include genomic DNA, cDNA, and RNA. The polynucleotide probe or primer can be RNA or DNA, and will comprise a portion of SEQ ID NO:1, the complement of SEQ ID NO:1, or an RNA equivalent thereof. Suitable assay methods in this regard include molecular genetic techniques known to those in the art, such as restriction fragment length polymorphism (RFLP) analysis, short tandem repeat (STR) analysis employing PCR techniques, ligation chain reaction (Barany, PCR Methods and Applications 1:5-16, 1991), ribonuclease protection assays, and other genetic linkage analysis techniques known in the art (Sambrook et al., ibid.; Ausubel et. al., ibid.; Marian, Chest 108:255-65, 1995). Ribonuclease protection assays (see, e.g., Ausubel et al., ibid., ch. 4) comprise the hybridization of an RNA probe to a patient RNA sample, after which the reaction product (RNA-RNA hybrid) is exposed to RNase. Hybridized regions of the RNA are protected from digestion. Within PCR assays, a patient's genetic sample is incubated with a pair of polynucleotide primers, and the region between the primers is amplified and recovered. Changes in size or amount of recovered product are indicative of mutations in the patient. Another PCR-based technique that can be employed is single strand conformational polymorphism (SSCP) analysis (Hayashi, PCR Methods and Applications 1:34-8, 1991).

The present invention also contemplates kits for performing a diagnostic assay for zacrp7 gene expression or to examine the zacrp7 locus. Such kits comprise nucleic acid probes, such as double-stranded nucleic acid molecules comprising the nucleotide sequence of SEQ ID NO:1, or a portion thereof, as well as single-stranded nucleic acid molecules having the complement of the nucleotide sequence of SEQ ID NO:1, or a portion thereof. Probe molecules may be DNA, RNA, oligonucleotides, and the like. Kits may comprise nucleic acid primers for performing PCR.

Such a kit can contain all the necessary elements to perform a nucleic acid diagnostic assay described above. A kit will comprise at least one container comprising a zacrp7 probe or primer. The kit may also comprise a second container comprising one or more reagents capable of indicating the presence of zacrp7 sequences. Examples of such indicator reagents include detectable labels such as radioactive labels, fluorochromes, chemiluminescent agents, and the like. A kit may also comprise a means for conveying to the user that the zacrp7 probes and primers are used to detect zacrp7 gene expression. For example, written instructions may state that the enclosed nucleic acid molecules can be used to detect either a nucleic acid molecule that encodes zacrp7, or a nucleic acid molecule having a nucleotide sequence that is complementary to a zacrp7-encoding nucleotide sequence. The written material can be applied directly to a container, or the written material can be provided in the form of a packaging insert.

Also contemplated is a method of detecting the presence of zacrp7 gene expression in a biological sample, comprising: (a) contacting a zacrp7 nucleic acid probe under hybridizing conditions with either (i) test RNA molecules isolated from the biological sample, or (ii) nucleic acid molecules synthesized from the isolated RNA molecules, wherein the probe consists of a nucleotide sequence comprising a portion of the nucleotide sequence of the nucleic acid molecule as described herein, or complements thereof, and (b) detecting the formation of hybrids of the nucleic acid probe and either the test RNA molecules or the synthesized nucleic acid molecules, wherein the presence of the hybrids indicates the presence of zacrp7 RNA in the biological sample.

Additionally provided is a method of detecting the presence of zacrp7 in a biological sample, comprising:(a) contacting the biological sample with an antibody, or an antibody fragment as described herein, wherein the contacting is performed under conditions that allow the binding of the antibody or antibody fragment to the biological sample, and (b) detecting any of the bound antibody or bound antibody fragment.

Zacrp7 polypeptides may be used in the analysis of energy efficiency of a mammal. Zacrp7 polypeptides found in serum or tissue samples may be indicative of a mammals ability to store food, with more highly efficient mammals tending toward obesity. More specifically, the present invention contemplates methods for detecting zacrp7 polypeptide comprising:

exposing a sample possibly containing zacrp7 polypeptide to an antibody attached to a solid support, wherein said antibody binds to an epitope of a zacrp7 polypeptide;

washing said immobilized antibody-polypeptide to remove unbound contaminants;

exposing the immobilized antibody-polypeptide to a second antibody directed to a second epitope of a zacrp7 polypeptide, wherein the second antibody is associated with a detectable label; and

detecting the detectable label. The concentration of zacrp7 polypeptide in the test sample appears to be indicative of the energy efficiency of a mammal. This information can aid nutritional analysis of a mammal. Potentially, this information may be useful in identifying and/or targeting energy deficient tissue.

A further aspect of the invention provides a method for studying insulin. Such methods of the present invention comprise incubating adipocytes in a culture medium comprising zacrp7 polypeptide, monoclonal antibody, agonist or antagonist thereof +insulin and observing changes in adipocyte protein secretion or differentiation.

Anti-microbial protective agents may be directly acting or indirectly acting. Such agents operating via membrane association or pore forming mechanisms of action directly attach to the offending microbe. Anti-microbial agents can also act via an enzymatic mechanism, breaking down microbial protective substances or the cell wall/membrane thereof. Anti-microbial agents, capable of inhibiting microorganism proliferation or action or of disrupting microorganism integrity by either mechanism set forth above, are useful in methods for preventing contamination in cell culture by microbes susceptible to that anti-microbial activity. Such techniques involve culturing cells in the presence of an effective amount of said zacrp7 polypeptide or an agonist or antagonist thereof.

Also, zacrp7 polypeptides or agonists thereof may be used as cell culture reagents in in vitro studies of exogenous microorganism infection, such as bacterial, viral or fungal infection. Such moieties may also be used in in vivo animal models of infection.

The present invention also provides methods of studying mammalian cellular metabolism. Such methods of the present invention comprise incubating cells to be studied, for example, human vascular endothelial cells, ±zacrp7 polypeptide, monoclonal antibody, agonist or antagonist thereof and observing changes in adipogenesis, gluconeogenesis, glycogenolysis, lipogenesis, glucose uptake, or the like.

Zacrp7 polypeptides, fragments, fusion proteins, antibodies, agonists or antagonists of the present invention can be used in methods for promoting blood flow within the vasculature of a mammal by reducing the number of platelets that adhere and are activated and the size of platelet aggregates. Used to such an end, zacrp7 can be administered prior to, during or following an acute vascular injury in the mammal. Vascular injury may be due to vascular reconstruction, including but not limited to, angioplasty, coronary artery bypass graft, microvascular repair or anastomosis of a vascular graft. Also contemplated are vascular injuries due to trauma, stroke or aneurysm. In other preferred methods the, vascular injury is due to plaque rupture, degradation of the vasculature, complications associated with diabetes and atherosclerosis. Plaque rupture in the coronary artery induces heart attack and in the cerebral artery induces stroke. Use of zacrp7 polypeptides, fragments, fusion proteins, antibodies, agonists or antagonists in such methods would also be useful for ameliorating whole system diseases of the vasculature associated with the immune system, such as disseminated intravascular coagulation (DIC) and SIDs. Additionally the complement inhibiting activity would be useful for treating non-vasculature immune diseases such as arteriolosclerosis. If desired, zacrp7 polypeptide, fragment, fusion protein, agonist, antagonist or antibody performance in this regard can be compared to proteins known to be functional in this regard, such as zsig37 or the like. In addition, zacrp7 polypeptides, fragments, fusion proteins, antibodies, agonists or antagonists may be evaluated in combination with one or more platelet aggregation or activation inhibiting agents to identify synergistic effects.

The polypeptides, fragments, fusion proteins, agonists, antagonists or antibodies may also be useful in treatments for acute vascular injury. Acute vascular injuries are those which occur rapidly (i.e. over days to months), in contrast to chronic vascular injuries (e.g. atherosclerosis) which develop over a lifetime. Acute vascular injuries often result from surgical procedures such as vascular reconstruction, wherein the techniques of angioplasty, endarterectomy, reduction atherectomy, endovascular stenting, endovascular laser ablation, anastomosis of a vascular graft or the like are employed. Hyperplasia may also occur as a delayed response in response to, e.g., emplacement of a vascular graft or organ transplantation.

A correlation has been found between the presence of C1q in localized ischemic myocardium and the accumulation of leukocytes following coronary occlusion and reperfusion. Release of cellular components following tissue damage triggers complement activation which results in toxic oxygen products that may be the primary cause of myocardial damage (Rossen et al., Circ. Res. 62:572-84, 1998 and Tenner, ibid.). Blocking the complement pathway was found to protect ischemic myocardium from reperfusion injury (Buerke et al., J. Pharm. Exp. Therp. 286:429-38, 1998). Proteins having complement inhibition and C1q binding activity would be useful for such purposes.

Collagen and C1q binding capabilities of adipocyte complement related protein homologs such as zacrp7 would be useful to pacify damaged collagenous tissues preventing platelet adhesion, activation or aggregation, and the activation of inflammatory processes which lead to the release of toxic oxygen products. By rendering the exposed tissue inert towards such processes as complement activity, thrombotic activity and immune activation, reduces the injurious effects of ischemia and reperfusion. In particular, such injuries would include trauma injury ischemia, intestinal strangulation, and injury associated with pre- and post-establishment of blood flow. Such polypeptides would be useful in the treatment of cardiopulmonary bypass ischemia and recesitation, myocardial infarction and post trauma vasospasm, such as stroke or percutanious transluminal angioplasty as well as accidental or surgical-induced vascular trauma.

Additionally such collagen- and Clq-binding polypeptides would be useful to pacify prosthetic biomaterials and surgical equipment to render the surface of the materials inert towards complement activation, thrombotic activity or immune activation. Such materials include, but are not limited to, collagen or collagen fragment-coated biomaterials, gelatin-coated biomaterials, fibrin-coated biomaterials, fibronectin-coated biomaterials, heparin-coated biomaterials, collagen and gel-coated stents, arterial grafts, synthetic heart valves, artificial organs or any prosthetic application exposed to blood that will bind zacrp7 at greater than 1×10⁸. Coating such materials can be done using methods known in the art, see for example, Rubens, U.S. Pat. No. 5,272,074.

Complement and C1q play a role in inflammation. The complement activation is initiated by binding of C1q to immunoglobulins (Johnston, Pediatr. Infect. Dis. J. 12:933-41, 1993; Ward and Ghetie, Therap. Immunol. 2:77-94, 1995). Inhibitors of C1q and complement would be useful as anti-inflammatory agents. Such application can be made to prevent infection. Additionally, such inhibitors can be administrated to an individual suffering from inflammation mediated by complement activation and binding of immune complexes to C1q. Inhibitors of Clq and complement would be useful in methods of mediating wound repair, enhancing progression in wound healing by overcoming impaired wound healing. Progression in wound healing would include, for example, such elements as a reduction in inflammation, fibroblasts recruitment, wound retraction and reduction in infection.

Ability of tumor cells to bind to collagen may contribute to the metastasis of tumors. Inhibitors of collagen binding are also useful for mediating the adhesive interactions and metastatic spread of tumors (Noeske-Jungbult et al., U.S. Pat. No. 5,723,312).

In addition, zacrp7 polypeptides, fragments, fusions agonists or antagonists thereof may be therapeutically useful for anti-microbial applications. For example, complement component C1q plays a role in host defense against infectious agents, such as bacteria and viruses. Clq is known to exhibit several specialized functions. For example, C1q triggers the complement cascade via interaction with bound antibody or C-reactive protein (CRP). Also, C1q interacts directly with certain bacteria, RNA viruses, mycoplasma, uric acid crystals, the lipid A component of bacterial endotoxin and membranes of certain intracellular organelles. C1q binding to the C1q receptor is believed to promote phagocytosis. C1q also appears to enhance the antibody formation aspect of the host defense system. See, for example, Johnston, Pediatr. Infect. Dis. J. 12(11): 933-41, 1993. Thus, soluble C1q-like molecules may be useful as anti-microbial agents, promoting lysis or phagocytosis of infectious agents.

The positively charged, extracellular, triple helix, collagenous domains of C1q and macrophage scavenger receptor were determined to play a role in ligand binding and were shown to have a broad binding specificity for polyanions (Acton et al., J. Biol. Chem. 268:3530-37, 1993). Lysophospholipid growth factor (lysophosphatidic acid, LPA) and other mitogenic anions localize at the site of damaged tissues and assist in wound repair. LPA exerts many biological effects including activation of platelets and up-regulation of matrix assembly. It is thought that LPA synergizes with other blood coagulation factors and mediates wound healing.

The collagenous domains of proteins such as C1q and macrophage scavenger receptor are know to bind acidic phospholipids such as LPA. The interaction of zacrp7 polypeptides, fragments, fusions, agonists or antagonists with mitogenic anions such as LPA can be determined using assays known in the art, see for example, Acton et al., ibid. Inhibition of inflammatory processes by polypeptides and antibodies of the present invention would also be useful in preventing infection at the wound site.

For pharmaceutical use, the proteins of the present invention can be formulated with pharmaceutically acceptable carriers for parenteral, oral, nasal, rectal, topical, transdermal administration or the like, according to conventional methods. In a preferred embodiment administration is made at or near the site of vascular injury. In general, pharmaceutical formulations will include a zacrp7 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton Pa., 19^{th} ed., 1995. Therapeutic doses will generally be determined by the clinician according to accepted standards, taking into account the nature and severity of the condition to be treated, patient traits, etc. Determination of dose is within the level of ordinary skill in the art.

As used herein a “pharmaceutically effective amount” of a zacrp7 polypeptide, fragment, fusion protein, agonist or antagonist is an amount sufficient to induce a desired biological result. The result can be alleviation of the signs, symptoms, or causes of a disease, or any other desired alteration of a biological system. For example, an effective amount of a zacrp7 polypeptide is that which provides either subjective relief of symptoms or an objectively identifiable improvement as noted by the clinician or other qualified observer. Such an effective amount of a zacrp7 polypeptide would provide, for example, inhibition of collagen-activated platelet activation and the complement pathway, including Clq, increase localized blood flow within the vasculature of a patient and/or reduction in injurious effects of ischemia and reperfusion. Modulation of inflammation associated with arthritis would include a reduction in inflammation and relief of pain or stiffness, in animal models, indications would be derived from macroscopic inspection of joints and change in swelling of hind paws. Effective amounts of the zacrp7 polypeptides can vary widely depending on the disease or symptom to be treated. The amount of the polypeptide to be administered and its concentration in the formulations, depends upon the vehicle selected, route of administration, the potency of the particular polypeptide, the clinical condition of the patient, the side effects and the stability of the compound in the formulation. Thus, the clinician will employ the appropriate preparation containing the appropriate concentration in the formulation, as well as the amount of formulation administered, depending upon clinical experience with the patient in question or with similar patients. Such amounts will depend, in part, on the particular condition to be treated, age, weight, and general health of the patient, and other factors evident to those skilled in the art. Typically a dose will be in the range of 0.01-100 mg/kg of subject. In applications such as balloon catheters the typical dose range would be 0.05-5 mg/kg of subject. Doses for specific compounds may be determined from in vitro or ex vivo studies in combination with studies on experimental animals. Concentrations of compounds found to be effective in vitro or ex vivo provide guidance for animal studies, wherein doses are calculated to provide similar concentrations at the site of action.

Polynucleotides encoding zacrp7 polypeptides are useful within gene therapy applications where it is desired to increase or inhibit zacrp7 activity. If a mammal has a mutated or absent zacrp7 gene, the zacrp7 gene can be introduced into the cells of the mammal. In one embodiment, a gene encoding a zacrp7 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zacrp7 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; WIPO Publication WO 95/07358; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zacrp7 gene transcription, such as to inhibit cell proliferation in vivo. Polynucleotides that are complementary to a segment of a zacrp7-encoding polynucleotide (e.g., a polynucleotide as set froth in SEQ ID NO:1) are designed to bind to zacrp7-encoding mRNA and to inhibit translation of such mRNA. Such antisense polynucleotides are used to inhibit expression of zacrp7 polypeptide-encoding genes in cell culture or in a subject.

Transgenic mice, engineered to express the zacrp7 gene, and mice that exhibit a complete absence of zacrp7 gene function, referred to as “knockout mice” (Snouwaert et al., Science 257:1083, 1992), may also be generated (Lowell et al., Nature 366:740-42, 1993). These mice may be employed to study the zacrp7 gene and the protein encoded thereby in an in vivo system.

The invention is further illustrated by the following non-limiting examples.

### EXAMPLE 1

### Identification of a Zacrp7 Sequence

The novel zacrp7 polypeptide encoding polynucleotide of the present invention was initially identified by querying an EST database for homologs of the adipocyte complement related proteins, characterized by a signal sequence, a collagen-like domain and a C1q domain. Polypeptides corresponding to ESTs meeting those search criteria were compared to known sequences to identify novel proteins having homology to this family. An assembled EST cluster was generated and predicted to be a secreted protein. The resulting 912 bp sequence is disclosed in SEQ ID NO: 1.

In order to isolate the polynucleotide of SEQ ID NO:1 from various tissues, probes and/or primers are designed from sequences disclosed herein such as SEQ ID NO:1. Tissues expressing zacrp7 could be identified either through hybridization (Northern Blots) or by reverse transcriptase (RT) PCR. Libraries are then generated from tissues which appear to show expression of zacrp7. Single clones from such libraries are then identified through hybridization with the probes and/or by PCR with the primers as described herein. Conformation of the zacrp7 cDNA sequence can be verified using the sequences provided herein.

### EXAMPLE 2

### Chromosomal Assignment and Placement of Zacrp7

Zacrp7 was mapped to chromosome 4 using the commercially available version of the Stanford G3 Radiation Hybrid Mapping Panel (Research Genetics, Inc., Huntsville, Ala.). The Stanford G3 RH Panel contains PCRable DNAs from each of 83 radiation hybrid clones of the whole human genome, plus two control DNAs (the RM donor and the A3 recipient). A publicly available WWW server (http://shgc-www.stanford.edu) allows chromosomal localization of markers.

For the mapping of zacrp7 with the Stanford G3 RH Panel, 20 μl reactions were set up in a 96-well microtiter plate (Stratagene, La Jolla, Calif.) and used in a RoboCycler Gradient 96 thermal cycler (Stratagene). Each of the 85 PCR reactions consisted of 2 μl 10× KlenTaq PCR reaction buffer (Clontech Laboratories, Inc., Palo Alto, Calif.), 1.6 μl dNTPs mix (2.5 mM each, Perkin-Elmer, Foster City, Calif.), 1 μl sense primer, ZC 23,631 (SEQ ID NO:12), 1 μl antisense primer ZC 23,632 (SEQ ID NO:13), 2 μl RediLoad (Research Genetics), 0.4 μl 50× Advantage KlenTaq Polymerase Mix (Clontech Laboratories, Inc.), 25 ng of DNA from an individual hybrid clone or control and ddH₂O for a total volume of 20 μl. The reactions were overlaid with an equal amount of mineral oil and sealed. The PCR cycler conditions were as follows: an initial 1 cycle 5 minute denaturation at 94° C., 35 cycles of a 45 seconds denaturation at 94° C., 45 seconds annealing at 64° C. and 1 minute AND 15 seconds extension at 72° C., followed by a final 1 cycle extension of 7 minutes at 72° C. The reactions were separated by electrophoresis on a 2% agarose gel (Life Technologies, Gaithersburg, Md.).

The results showed linkage of zacrp7 to the chromosome 4 marker SHGC-35585 with a LOD score of >16 and at a distance of 0 cR_{—}10000 from the marker. The use of surrounding genes/markers positions zacrp7 in the 4p15 chromosomal region.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide comprising a sequence of amino acid residues that is at least 80% identical in amino acid sequence to residues 52-303 of SEQ ID NO:2, wherein said sequence comprises:
Gly-Xaa-Xaa and Gly-Xaa-Pro collagen repeats forming a collagen-like domain, wherein Xaa is any amino acid residue; and
a carboxyl-terminal C1q domain.

2. An isolated polypeptide according to claim 1, wherein said polypeptide is at least 90% identical in amino acid sequence to residues 31-303 of SEQ ID NO:2.

3. An isolated polypeptide according to claim 2, wherein any differences between said polypeptide and SEQ ID NO:2 are due to conservative amino acid substitutions.

4. An isolated polypeptide according to claim 2, wherein said collagen-like domain consists of 26 Gly-Xaa-Xaa collagen repeats and 8 Gly-Xaa-Pro collagen repeats.

5. An isolated polypeptide according to claim 2, wherein said polypeptide comprises:
an amino terminal region;
26 Gly-Xaa-Xaa collagen repeats and 8 Gly-Xaa-Pro collagen repeats forming a collagen-like domain, wherein Xaa is any amino acid residue; and
a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2.

6. An isolated polypeptide according to claim 2, wherein said polypeptide specifically binds with an antibody that specifically binds with a polypeptide of SEQ ID NO:2.

7. An isolated polypeptide according to claim 2, wherein said collagen-like domain comprises amino acid residues 52-153 of SEQ ID NO:2.

8. An isolated polypeptide according to claim 2, wherein said C1q domain comprises amino acid residues 154-303 of SEQ ID NO:2.

9. An isolated polypeptide according to claim 1, wherein said polypeptide comprises residues 52-303 of SEQ ID NO:2.

10. An isolated polypeptide according to claim 2, wherein said polypeptide comprises residues 31-303 of SEQ ID NO:2.

11. An isolated polypeptide according to claim 2, wherein said polypeptide comprises residues 1-303 of SEQ ID NO:2.

12. An isolated polypeptide according to claim 1, wherein said polypeptide is complexed by intermolecular disulfide bonds to form a homotrimer.

13. An isolated polypeptide according to claim 1 wherein said polypeptide is complexed by intermolecular disulfide bonds, to one or more polypeptides having a collagen-like domain, to form a heterotrimer.

14. An isolated polypeptide according to claim 1, covalently linked at the amino or carboxyl terminus to a moiety selected from the group consisting of affinity tags, toxins, radionucleotides, enzymes and fluorophores.

15. An isolated polypeptide selected from the group consisting of:
a) a polypeptide consisting of a sequence of amino acid residues from residue 52 to residue 153 of SEQ ID NO:2; and
b) a polypeptide consisting of a sequence of amino acid residues from residue 154 to residue 303 of SEQ ID NO:2.

16. A fusion protein consisting essentially of a first portion and a second portion joined by a peptide bond, said first portion consisting of a polypeptide selected from the group consisting of:
a) polypeptide according to claim 1;
b) polypeptide comprising: an amino terminal region; 26 Gly-Xaa-Xaa collagen repeats and 8 Gly-Xaa-Pro collagen repeats forming a collagen-like domain, wherein Xaa is any amino acid residue; and a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2;
c) a portion of the zacrp7 polypeptide as shown in SEQ ID NO:2, comprising the collagen-like domain or a portion of the collagen-like domain capable of trimerization or oligomerization;
d) a portion of the zacrp7 polypeptide as shown in SEQ ID NO:2, comprising the C1q domain or an active portion of the C1q domain; or
e) a portion of the zacrp7 polypeptide as shown in SEQ ID NO:2 comprising of the collagen-like domain and the C1q domain; and
said second portion comprising another polypeptide.

17. A fusion protein according to claim 16, wherein said first portion is selected from the group consisting of:
a) a polypeptide consisting of the sequence of amino acid residue 52 to amino acid residue 153 of SEQ ID NO:2;
b) a polypeptide consisting of the sequence of amino acid residue 154 to amino acid residue 303 of SEQ ID NO:2;
c) a polypeptide consisting of the sequence of amino acid residue 52 to 303 of SEQ ID NO:2;
d) a polypeptide consisting of the sequence of amino acid residue 31 to 303 of SEQ ID NO:2; and
e) a polypeptide consisting of the sequence of amino acid residue 1 to 303 of SEQ ID NO:2.

18. A polypeptide according to claim 1; in combination with a pharmaceutically acceptable vehicle.

19. A method of producing an antibody to a polypeptide comprising:
inoculating an animal with a polypeptide selected from the group consisting of:
a) polypeptide according to claim 1;
b) polypeptide comprising: an amino terminal region; 26 Gly-Xaa-Xaa collagen repeats and 8 Gly-Xaa-Pro collagen repeats forming a collagen-like domain, wherein Xaa is any amino acid residue; and a carboxyl-terminal Clq domain comprising 10 beta strands corresponding to amino acid residues 164-168, 184-186, 192-195, 199-201, 205-216, 220-226, 231-238, 241-253, 258-263 and 281-285 of SEQ ID NO:2;
c) a portion of the zacrp7 polypeptide as shown in SEQ ID NO:2, comprising the collagen-like domain or a portion of the collagen-like domain capable of trimerization or oligomerization;
d) a portion of the zacrp7 polypeptide as shown in SEQ ID NO:2, comprising the C1q domain or an active portion of the C1q domain; or
e) a portion of the zacrp7 polypeptide as shown in SEQ ID NO:2 comprising of the collagen-like domain and the Clq domain; and
wherein said polypeptide elicits an immune response in the animal to produce the antibody; and
isolating the antibody from the animal.

20. An antibody or antibody fragment that specifically binds to a polypeptide according to claim 1.

21. An antibody according to claim 20, wherein said antibody is selected from the group consisting of:
a) polyclonal antibody;
b) murine monoclonal antibody;
c) humanized antibody derived from b); and
d) human monoclonal antibody.

22. An antibody fragment according to claim 20, wherein said antibody fragment is selected from the group consisting of F(ab′), F(ab), Fab′, Fab, Fv, scFv, and minimal recognition unit.

23. An anti-idiotype antibody that specifically binds to said antibody of claim 20.

24 A binding protein that specifically binds to an epitope of a polypeptide according the claim 1.

25. An isolated polynucleotide encoding a polypeptide comprising a sequence of amino acid residues that is at least 80% identical in amino acid sequence to residues 52-153 of SEQ ID NO:2, wherein said sequence comprises:
Gly-Xaa-Xaa and Gly-Xaa-Pro collagen repeats forming a collagen-like domain, wherein Xaa is any amino acid residue; and
a carboxyl-terminal C1q domain.

26. An isolated polynucleotide according to claim 25, wherein said polypeptide is at least 90% identical in amino acid sequence to residues 31-303 of SEQ ID NO:2.

27. An isolated polynucleotide according to claim 25, wherein said collagen-like domain consists of 26 Gly-Xaa-Xaa collagen repeats and 8 Gly-Xaa-Pro collagen repeats.

28. An isolated polynucleotide according to claim 25, wherein said polypeptide comprises:
an amino terminal region;
26 Gly-Xaa-Xaa collagen repeats and 8 Gly-Xaa-Pro collagen repeats forming a collagen-like domain, wherein Xaa is any amino acid residue; and
a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2.

29. An isolated polynucleotide according to claim 25, wherein any differences between said polypeptide and SEQ ID NO:2 are due to conservative amino acid substitutions.

30. An isolated polynucleotide according to claim 25, wherein said polypeptide specifically binds with an antibody that specifically binds with a polypeptide of SEQ ID NO:2.

31. An isolated polynucleotide according to claim 25, wherein said collagen-like domain comprises amino acid residues 52-153 of SEQ ID NO:2.

32. An isolated polynucleotide according to claim 25, wherein said polypeptide comprises residues 52-303 of SEQ ID NO:2.

33. An isolated polynucleotide according to claim 25, wherein said polypeptide comprises residues 31-303 of SEQ ID NO:2.

34. An isolated polynucleotide according to claim 25, wherein said polypeptide comprises residues 1-303 of SEQ ID NO:2.

36. An isolated polynucleotide according to claim 25, wherein said polypeptide is covalently linked at the amino or carboxyl terminus to a moiety selected from the group consisting of affinity tags, toxins, radionucleotides, enzymes and fluorophores.

37. An isolated polynucleotide selected from the group consisting of,
a) a sequence of nucleotides from nucleotide 1 to nucleotide 909 of SEQ ID NO:1;
b) a sequence of nucleotides from nucleotide 91 to nucleotide 909 of SEQ ID NO:1;
c) a sequence of nucleotides from nucleotide 91 to nucleotide 459 of SEQ ID NO:1;
d) a sequence of nucleotides from nucleotide 154 to nucleotide 909 of SEQ ID NO:1;
e) a sequence of nucleotides from nucleotide 154 to nucleotide 459 of SEQ ID NO:1;
f) a sequence of nucleotides from nucleotide 460 to nucleotide 909 of SEQ ID NO:1;
g) a polynucleotide encoding a polypeptide consisting of the amino acid sequence of residues 51 to 153 of SEQ ID NO:2;
h) a polynucleotide encoding a polypeptide consisting of the amino acid sequence of residues 154 to 303 of SEQ ID NO:2;
i) a polynucleotide that remains hybridized, following stringent wash conditions, to a polynucleotide consisting of the nucleotide sequence of SEQ ID NO:1, or the complement of SEQ ID NO:1;
j) nucleotide sequences complementary to a), b), c), d), e), f), g), h) or i) and
k) degenerate nucleotide sequences of g) or h).

38. An isolated polynucleotide encoding a fusion protein consisting essentially of a first portion and a second portion joined by a peptide bond,
said first portion consisting of a polypeptide selected from the group consisting of:
a) polypeptide according to claim 1;
b) polypeptide comprising: an amino terminal region; 26 Gly-Xaa-Xaa collagen repeats and 8 Gly-Xaa-Pro collagen repeats forming a collagen-like domain, wherein Xaa is any amino acid residue; and a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2;
c) a portion of the zacrp7 polypeptide as shown in SEQ ID NO:2, comprising the collagen-like domain or a portion of the collagen-like domain capable of trimerization or oligomerization;
d) a portion of the zacrp7 polypeptide as shown in SEQ ID NO:2, comprising the C1q domain or an active portion of the C1q domain; or
e) a portion of the zacrp7 polypeptide as shown in SEQ ID NO:2 comprising of the collagen-like domain and the C1q domain; and
said second portion comprising another polypeptide.

39. An isolated polynucleotide consisting of the sequence of nucleotide 1 to nucleotide 909 of SEQ ID NO:11.

40. An expression vector comprising the following operably linked elements:
a transcription promoter;
a DNA segment encoding a polypeptide according to claim 1; and
a transcription terminator.

41. An expression vector according to claim 40, wherein said DNA segment encodes a polypeptide that is at least 90% identical in amino acid sequence to residues 31-303 of SEQ ID NO:2.

42. An expression vector according to claim 40, wherein said collagen-like domain consists of 26 Gly-Xaa-Xaa collagen repeats and 8 Gly-Xaa-Pro collagen repeats.

43. An expression vector according to claim 40, wherein said DNA segment encodes a polypeptide comprising:
an amino terminal region;
26 Gly-Xaa-Xaa collagen repeats and 8 Gly-Xaa-Pro collagen repeats forming a collagen-like domain, wherein Xaa is any amino acid residue; and
a carboxyl-terminal C1q domain comprising 10 beta strands corresponding to amino acid residues 164-168, 184-186, 192-195, 199-201, 205-216, 220-226, 231-238, 241-253, 258-263 and 281-285 of SEQ ID NO:2.

44. An expression vector according to claim 40, wherein said collagen-like domain comprises amino acid residues 52-153 of SEQ ID NO:2.

45. An expression vector according to claim 40, wherein any differences between said polypeptide and SEQ ID NO:2 are due to conservative amino acid substitutions.

46. An expression vector according to claim 40, wherein said polypeptide specifically binds with an antibody that specifically binds with a polypeptide of SEQ ID NO:2.

47. An expression vector according to claim 40, wherein said DNA encodes a polypeptide comprising residues 52-303 of SEQ ID NO:2.

48. An expression vector according to claim 40, wherein said DNA segment encodes a polypeptide comprising residues 31-303 of SEQ ID NO:2.

49. An expression vector according to claim 40, wherein said DNA segment encodes a polypeptide comprising residues 1-303 of SEQ ID NO:2.

50. An expression vector according to claim 40, wherein said DNA segment further encodes a secretory signal sequence operably linked to said polypeptide.

51. An expression vector according the claim 40, wherein said secretory signal sequence comprises residues 1-30 of SEQ ID NO:2.

52. A cultured cell into which has been introduced an expression vector according to claim 40, wherein said cell expresses said polypeptide encoded by said DNA segment.

53. A cultured cell according to claim 52, which further includes one or more expression vectors comprising DNA segments encoding polypeptides having collagen-like domains.

54. A method of producing a protein comprising:
culturing a cell into which has been introduced an expression vector according to claim 40;
whereby said cell expresses said protein encoded by said DNA segment; and
recovering said expressed protein.

55. A method of producing a protein according to claim 54, wherein said expressed protein is a homotrimer.

56. A method of producing a protein according to claim 54, wherein said expressed protein is a heterotrimer.

57. A method of detecting the presence of zacrp7 gene expression in a biological sample, comprising:
(a) contacting a zacrp7 nucleic acid probe under hybridizing conditions with either (i) test RNA molecules isolated from the biological sample, or (ii) nucleic acid molecules synthesized from the isolated RNA molecules, wherein the probe consists of a nucleotide sequence comprising a portion of the nucleotide sequence of the nucleic acid molecule of claim 25, or complements thereof, and
(b) detecting the formation of hybrids of the nucleic acid probe and either the test RNA molecules or the synthesized nucleic acid molecules,
wherein the presence of the hybrids indicates the presence of zacrp7 RNA in the biological sample.

58. A method of detecting the presence of zacrp7 in a biological sample, comprising:
(a) contacting the biological sample with an antibody, or an antibody fragment, of claim 20, wherein the contacting is performed under conditions that allow the binding of the antibody or antibody fragment to the biological sample, and
(b) detecting any of the bound antibody or bound antibody fragment.

59. An isolated polypeptide comprising amino acid residues 154-303 of SEQ ID NO:2.

60. An isolated polypeptide according to claim 59, comprising amino acid residues 52-303 of SEQ ID NO:2.

61. An isolated polypeptide according to claim 60, comprising amino acid residues 31-303 of SEQ ID NO:2.

62. An isolated polypeptide according to claim 61, comprising amino acid residues 1-303 of SEQ ID NO:2.

63. An isolated polypeptide comprising amino acid residues 52-153 of SEQ ID NO:2.

64. An isolated polypeptide according to claim 59, wherein said polypeptide is complexed by intermolecular disulfide bonds to form a homotrimer.

65. An isolated polypeptide according to claim 59, wherein said polypeptide is complexed by at least one intermolecular disulfide bond to one or more polypeptides having a collagen-like domain to form a heterotrimer.

66. An isolated polypeptide according to claim 59, wherein the polypeptide is covalently linked at the amino or carboxyl terminus to a moiety selected from the group consisting of affinity tags, toxins, radionucleotides, enzymes and fluorophores.

67. An isolated polypeptide comprising a sequence selected from the group consisting of:
a) amino acid residues 52-153 of SEQ ID NO:2;
b) amino acid residues 154-303 of SEQ ID NO:2;
c) amino acid residues 52-303 of SEQ ID NO:2;
d) amino acid residues 31-303 of SEQ ID NO:2; and
e) amino acid residues 1-303 of SEQ ID NO:2.

68. A fusion protein comprising a first portion and a second portion joined by a peptide bond, wherein said first portion is an isolated polypeptide comprising amino acid residues 154-303 of SEQ ID NO:2 or an isolated polypeptide comprising amino acid residues 52-153 of SEQ ID NO:2, and wherein said second portion comprises another polypeptide.

69. A fusion protein according to claim 68, wherein said first portion is selected from the group consisting of:
a) a polypeptide comprising amino acid residues 52 to 303 of SEQ ID NO:2;
b) a polypeptide comprising amino acid residues 31 to 303 of SEQ ID NO:2; and
c) a polypeptide comprising amino acid residues 1 to 303 of SEQ ID NO:2.

70. A polypeptide according to claim 59; in combination with a pharmaceutically acceptable vehicle.

71. A method of producing an antibody to a polypeptide, the method comprising:
inoculating an animal with a polypeptide according to claim 59;
wherein said polypeptide elicits an immune response in the animal to produce the antibody; and
isolating the antibody from the animal.

72. An antibody or antibody fragment that specifically binds to a polypeptide according to claim 59.

73. An antibody according to claim 72, wherein said antibody is selected from the group consisting of:
a) polyclonal antibody;
b) murine monoclonal antibody;
c) humanized antibody derived from b); and
d) human monoclonal antibody.

74. An antibody fragment according to claim 72, wherein said antibody fragment is selected from the group consisting of F(ab′), F(ab), Fab′, Fab, Fv, scFv, and minimal recognition unit.

75. An anti-idiotype antibody that specifically binds to said antibody of claim 72.

76. An isolated polynucleotide encoding a polypeptide comprising amino acid residues 154-303 of SEQ ID NO:2.

77. An isolated polynucleotide encoding a polypeptide according to claim 76, comprising amino acid residues 52-303 of SEQ ID NO:2.

78. An isolated polynucleotide encoding a polypeptide according to claim 77, comprising amino acid residues 31-303 of SEQ ID NO:2.

79. An isolated polynucleotide encoding a polypeptide according to claim 77, comprising amino acid residues 1-303 of SEQ ID NO:2.

80. An isolated polynucleotide encoding a polypeptide comprising amino acid residues 15-153 of SEQ ID NO:2.

81. An isolated polynucleotide encoding a polypeptide according to claim 76, wherein said polypeptide is complexed by at least one intermolecular disulfide bond to form a homotrimer.

82. An isolated polynucleotide encoding a polypeptide according to claim 76, wherein said polypeptide is complexed by at least one intermolecular disulfide bond to one or more polypeptides having a collagen-like domain to form a heterotrimer.

83. An isolated polynucleotide encoding a polypeptide according to claim 76, wherein said polypeptide is covalently linked at the amino or carboxyl terminus to a moiety selected from the group consisting of affinity tags, toxins, radionucleotides, enzymes and fluorophores.

84. An isolated polynucleotide encoding a polypeptide comprising a sequence selected from the group consisting of:
a) amino acid residues 52-153 of SEQ ID NO:2;
b) amino acid residues 154-303 of SEQ ID NO:2;
c) amino acid residues 52-303 of SEQ ID NO:2;
d) amino acid residues 31-303 of SEQ ID NO:2; and
e) amino acid residues 1-303 of SEQ ID NO:2.

85. An isolated polynucleotide encoding a fusion protein comprising a first portion and a second portion joined by a peptide bond, wherein said first portion is an isolated polypeptide comprising amino acid residues 154-303 of SEQ ID NO:2 or an isolated polypeptide comprising amino acid residues 52-153 of SEQ ID NO:2, and wherein said second portion comprises another polypeptide.

86. An isolated polynucleotide encoding a fusion protein according to claim 85, wherein said first portion is selected from the group consisting of:
a) a polypeptide comprising amino acid residues 52 to 303 of SEQ ID NO:2;
b) a polypeptide comprising amino acid residues 31 to 303 of SEQ ID NO:2; and
c) a polypeptide comprising amino acid residues 1 to 303 of SEQ ID NO:2.

87. An isolated polynucleotide comprising a sequence selected from the group consisting of,
a) nucleotides 1 to 909 of SEQ ID NO:1;
b) nucleotides 91 to 909 of SEQ ID NO:1;
c) nucleotides 154 to 909 of SEQ ID NO:1;
d) nucleotides 460 to 909 of SEQ ID NO:1; and
e) nucleotide sequences complementary to a), b), c), or d).

88. An expression vector comprising the following operably linked elements:
a transcription promoter;
a DNA segment encoding a polypeptide according to claim 59; and
a transcription terminator.

89. An expression vector according to claim 88, wherein said DNA segment encodes a polypeptide comprising amino acid residues 52-303 of SEQ ID NO:2.

90. An expression vector according to claim 88, wherein said DNA segment encodes a polypeptide comprising amino acid residues 31-303 of SEQ ID NO:2.

91. An expression vector according to claim 88, wherein said DNA segment encodes a polypeptide comprising amino acid residues 1-303 of SEQ ID NO:2.

92. An expression vector according to claim 88, wherein said DNA segment further encodes a secretory signal sequence operably linked to said polypeptide.

93. An expression vector according the claim 92, wherein said secretory signal sequence comprises amino acid residues 1-30 of SEQ ID NO:2.

94. An expression vector comprising the following operably linked elements:
a transcription promoter;
a DNA segment encoding a polypeptide comprising amino acid residues 52-153 of SEQ ID NO:2; and
a transcription terminator.

95. A cultured cell into which has been introduced an expression vector according to claim 88, wherein said cell expresses said polypeptide encoded by said DNA segment.

96. A cultured cell according to claim 95, further comprising one or more expression vectors comprising DNA segments encoding polypeptides having collagen-like domains.

97. A method of producing a protein comprising:
culturing a cell into which has been introduced an expression vector according to claim 88, wherein said cell expresses said protein encoded by said DNA segment; and

98. The method of claim 97 further comprising recovering said expressed protein.

99. A method of producing a protein according to claim 97, wherein said expressed protein is a homotrimer.

100. A method of producing a protein according to claim 97, wherein said expressed protein is a heterotrimer.
